(19) United States
(12) Patent Application Publication
(10) Pub. No.: US 2026/0256679 A1
(43) Pub. Date: Sep. 3, 2026

(54) HAIR TREATMENT COMPOSITION

(71) Applicant: L'OREAL, Paris (FR)

(72) Inventors: Krishna R. RAGHUPATHI, Morganville, NJ (US); Manon CHAUMONTET, Jersey City, NJ (US); Lynne MCCULLOUGH, Brooklyn, NY (US); Marvin John DUROGENE, Neptune, NJ (US)

(73) Assignee: L'OREAL, Paris (FR)

(21) Appl. No.: 19/066,812

(22) Filed: Feb. 28, 2025

(51) Int. Cl. A61K 8/85; A61K 8/06; A61K 8/34; A61K 8/36; A61K 8/92; A61Q 5/00; A61Q 5/12

(52) U.S. Cl. CPC A61K 8/85; A61K 8/062; A61K 8/345; A61K 8/361; A61K 8/922; A61Q 5/002; A61Q 5/12; A61K 2800/30; A61K 2800/5426; A61K 2800/594; A61K 2800/805

(57) ABSTRACT

A hair treatment composition comprising: (a) one or more cationic amidoamine surfactants; (b) one or more sebacic acid copolymers; (c) one or more vegetable oils; (d) one or more fatty acids; (e) optionally, one or more fatty alcohols; (f) one or more polyols with 2 to 8 carbon atoms; (g) optionally, one or more cationic polymers; and (h) water, is disclosed. The hair treatment composition is particularly useful in method for improving body, volume, frizz control, softness, and smoothness of hair.

### FIELD OF THE DISCLOSURE

The instant disclosure is drawn to hair treatment compositions containing a sebacic acid copolymer, and to methods for treating hair, for example, conditioning, managing, and improving the look and feel of hair.

### BACKGROUND

Many individuals suffer from dry and damaged hair. Dryness and damage can occur due to several factors including weather exposure, mechanical treatments (e.g., brushing hair), excessive treatments using chemicals, dying hair, heat styling, etc. In combination, using cleansing products that can be excessively stripping of hair's natural oils can also not only lead to split ends and dull hair, but also exacerbate dry hair. To mitigate the damage, oil treatments, conditioner, hair masks, and chemical treatments are commonly used.

The popularity and usage of oils for dry hair treatments has increased due to their effectiveness and simplicity. Commonly used oils include olive oil, mineral oil, avocado oil, apricot kernel oil, rice bran oil, and coconut oil. However, one problem is that effects are not usually seen after more than several hours (e.g., 8 hours) of treatment and several treatments are usually required, making it time consuming and labor intensive.

Individuals desire a treatment for hair or damaged hair that is not time consuming and labor intensive to use. A variety of approaches have been developed to condition the hair. A common method of providing conditioning benefit is through using conditioning agents such as cationic surfactants and polymers, high melting point fatty compounds, low melting point oils, silicone compounds, and combinations thereof. Most of these conditioning agents are known to provide various conditioning benefits.

However, there is still a need for providing improved hair manageability, for example, improved hair alignment, reduced unwanted volume (especially reduced frizz), and increased shine. There is also a need to develop hair care products that can impart other benefits at the same time in addition to caring and conditioning benefits, such as styling, volume, shaping, curl definition (for curly or wavy hair), and restyle-ability or reshaping (without the need to reapply the product).

### SUMMARY OF THE DISCLOSURE

There is a need for new and improved products and methods for treating curly or wavy hair, specifically for achieving visible and lasting curl definition, elongation, and maintenance, particularly under humid conditions. The ideal composition should not only provide good curl definition and regularity but also enhance the hair with additional cosmetic benefits such as improved body, volume, frizz control, softness, and smoothness. Moreover, the composition should be non-irritating, non-toxic, and long-lasting.

The present disclosure addresses these needs with a unique hair treatment composition and corresponding methods for conditioning, softening, and reducing frizz. A key feature of the composition is the inclusion of one or more sebacic acid copolymers, which are particularly effective in delivering long-lasting cosmetic benefits to the hair. The hair treatment composition includes:
(a) one or more cationic amidoamine surfactants;
(b) one or more sebacic acid copolymers;
(c) one or more vegetable oils;
(d) one or more fatty acids;
(e) optionally, one or more fatty alcohols;
(f) one or more polyols with 2 to 8 carbon atoms;
(g) optionally, one or more cationic polymers; and
(h) water;
   wherein the hair treatment composition is an oil-in-water emulsion.

Nonlimiting examples of cationic amidoamine surfactants include oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitoamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, stearamidoethyldiethylamine, or a combination thereof.

Nonlimiting examples of sebacic acid copolymers include capryloyl glycerin/sebacic acid copolymer, hydrogenated castor oil/sebacic acid copolymer, polyhydroxybutyrate-sebacate copolymer, acrylates/sebacic acid copolymer, polycaprolactone/sebacic acid copolymer, and polylactic acid/sebacic acid copolymer. Preferably, the sebacic acid copolymer is capryloyl glycerin/sebacic acid copolymer, hydrogenated castor oil/sebacic acid copolymer, or a combination thereof.

Hydrogenated castor oil/sebacic acid copolymer is a biodegradable polymer derived from natural sources. It is synthesized by polymerizing hydrogenated castor oil with sebacic acid (a dicarboxylic acid derived from castor oil). It includes long-chain aliphatic structures, providing flexibility, hydrophobicity, and durability.

Nonlimiting examples of vegetable oils include castor oil, arachis oil, amaranth seed oil, apricot oil, argan oil, avocado oil, babassu oil, cottonseed oil, borage seed oil, camelina oil, camellia oil, safflower oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, manila oil, evening primrose oil, olive oil, palm oil, palm kernel oil, brazil nut oil, pecan nut oil, peach kernel oil, rapeseed oil, rosehip oil, sea buckthorn fruit oil, sea buckthorn kernel oil, sesame oil, soybean oil, sunflower oil, grapeseed oil, walnut oil, wild rose oil, wheat germ oil, coconut oil, avocado oil, arachis oil, apricot oil, argan oil, and babassu oil. In a preferred embodiment, the hair treatment composition includes castor oil.

Fatty acids are a type of organic compound composed of a long hydrocarbon chain with a carboxyl group (—COOH) at one end. Nonlimiting examples of fatty acids include those with 10 to 24 carbon atoms in the hydrocarbon chain, wherein the chain may be straight or branched, and saturated or unsaturated. More specific but nonlimiting examples include myristic acid, lauric acid, palmitic acid, stearic acid, behenic acid, arichidonic acid, oleic acid, isostearic acid, and sebacic acid.

The hair treatment compositions optionally include one or more fatty alcohols. A fatty alcohol is a type of organic compound consisting of a long hydrocarbon chain with a hydroxyl (—OH) group at one end. Nonlimiting examples of fatty alcohols include those with 10 to 24 carbon atoms in the hydrocarbon chain, wherein the chain may be straight or branched, and saturated or unsaturated. The one or more fatty alcohol may be selected from saturated or unsaturated, linear or branched fatty alcohols with 10 to 30 carbon atoms, preferably from 12 to 28 carbon atoms, more preferably from 14 to 24 carbon atoms. Nonlimiting examples of fatty alcohols include octyldodecanol, decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a combination thereof. In some instances, the hair treatment compositions preferably include octyldodecanol, oleyl alcohol, behenyl alcohol, cetearyl alcohol, or combinations thereof. In another embodiment, the one or more fatty alcohols are selected from octyldodecanol, decyl alcohol, undecyl alcohol, dodecyl alcohol, myristyl alcohol, cetearyl alcohol, cetyl alcohol, stearyl alcohol, isostearyl alcohol, isocetyl alcohol, behenyl alcohol, linalool, oleyl alcohol, isotridecyl alcohol, and myricyl alcohol.

The hair treatment composition includes one or more polyols with 2 to about 8 carbon atoms. Nonlimiting examples of polyols with 2 to 8 carbon atoms are selected from ethylene glycol, propylene glycol, butylene glycol, hexylene glycol, pentylene glycol, 1,3-propanediol, diethylene glycol, dipropylene glycol, caprylyl glycol, and glycerin.

The hair treatment composition optionally includes one or more cationic polymers. These polymers carry a positive charge (cationic), which allows them to bind to the negatively charged surfaces of the skin and hair. Nonlimiting examples of cationic polymers include cationic polysaccharides derivatives (cationic polysaccharides), cationic gum derivatives (cationic gums), polyquaterniums, polymer derivatives of diallyldimethyl ammonium chloride, polymer derivatives of methacrylamidopropyltrimethylammonium chloride, cationic cellulose derivatives (cationic celluloses), quaternized hydroxyethyl cellulose, cationic starch derivatives (cationic starches), cationic guar gum derivatives (cationic guars), copolymers of acrylamide and dimethyldiallyammonium chloride, and a mixture thereof.

Nonlimiting examples of preferred cationic polymers include cationic polysaccharides, for example, cationic polysaccharide selected from cationic celluloses, cationic starches, cationic dextrans, and cationic guars. In a preferred embodiment, the hair treatment composition includes one or more cationic guars. Nonlimiting examples of cationic guars include guar hydroxypropyltrimonium chloride, hydroxypropyl guar hydroxypropyltrimonium chloride, guar dimethylhydroxypropyltrimonium chloride, and guar hydroxypropyltrimonium methylsulfate.

In a preferred embodiment, the hair treatment composition includes one or more cationic guars. Nonlimiting examples of cationic guars include guar hydroxypropyltrimonium chloride, hydroxypropyl guar hydroxypropyltrimonium chloride, guar dimethylhydroxypropyltrimonium chloride, and guar hydroxypropyltrimonium methylsulfate.

The hair treatment composition optionally includes one or more nonionic polymers, one or more anionic polymers, or combinations thereof.

The hair treatment composition may be applied to wet or damp hair. It can serve as a replacement for conditioner when applied after shampooing. Alternatively, it can be used following both shampooing and conditioning, with the option to rinse it out or leave it on, functioning as either a rinse-off or leave-in product. In some cases, the hair treatment composition is applied as a leave-on treatment; the product is applied to the hair and allowed to remain on the hair without rinsing before styling the hair.

### DETAILED DESCRIPTION OF THE DISCLOSURE

This hair treatment composition of the instant case improves the appearance, feel, and manageability of hair, leveraging the benefits of one or more sebacic acid copolymers, fatty acids, and cationic amidoamine surfactants. The hair treatment composition is particularly useful for treating curly or wavy hair, specifically for achieving visible and lasting curl definition, elongation, and maintenance, particularly under humid conditions. Treatment with the composition not only provides good curl definition and regularity to hair, but also enhance the hair with additional cosmetic benefits such as improved body, volume, frizz control, softness, and smoothness. Finally, the hair treatment composition is non-irritating, non-toxic, and its benefits are long-lasting.

The hair treatment composition typically includes:
(a) about 0.1 to about 8 wt. % of one or more cationic amidoamine surfactants;
(b) about 0.1 to about 5 wt. % of one or more sebacic acid copolymers;
(c) about 0.1 to about 10 wt. % of one or more vegetable oils;
(d) about 0.1 to about 10 wt. % of one or more fatty acids;
(e) optionally, one or more fatty alcohols;
(f) about 5 to about 25 wt. % of one or more polyols with 2 to 8 carbon atoms;
(g) optionally, about 0.02 to about 5 wt. % of one or more cationic polymers; and
(h) water;
   wherein the hair treatment composition is an oil-in-water emulsion, and all percentages by weight are based on a total weight of the hair treatment composition.

### (a) Cationic Amidoamine Surfactants

The term “cationic amidoamine surfactant” is an amidoamine surfactant that may be positively charged when it is contained in the compositions according to the instant disclosure. This surfactant may bear one or more positive permanent charges or may contain one or more functional groups that are cationizable.

Nonlimiting examples of cationic amidoamine surfactants include oleamidopropyl dimethylamine, stearamidopropyl dimethylamine, isostearamidopropyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, stearamidoethyldiethylamine, and combinations thereof.

The total amount of the one or more cationic surfactants in the hair treatment compositions will vary but is typically from about 0.1 to about 10 wt. %, based on the total weight of the hair treatment composition. In further embodiments, the hair treatment composition includes from about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, about 1 to about 3 wt. %, about 2 to about 10 wt. %, about 2 to about 8 wt. %, about 2 to about 6 wt. %, about 2 to about 5 wt. %, or about 2 to about 4 wt. % of the one or more cationic surfactants, based on a total weight of the hair treatment composition. Preferably, the hair treatment composition includes from about 0.5 to about 8 wt. %, more preferably about 1 to about 6 wt. %, and even more preferably, about 2 to about 5 wt. % of one or more cationic surfactants, based on the total weight of the hair treatment composition.

### (b) Sebacic Acid Copolymers

Sebacic acid copolymers are polymers that incorporate sebacic acid, a 10-carbon dicarboxylic acid, into their structure through copolymerization with one or more different monomers (often diols or other acids). Nonlimiting examples of sebacic acid copolymers include capryloyl glycerin/sebacic acid copolymer, hydrogenated castor oil/sebacic acid copolymer, polyhydroxybutyrate-sebacate copolymer, acrylates/sebacic acid copolymer, polycaprolactone/sebacic acid copolymer, polylactic acid/sebacic acid copolymer, poly(sebacic acid-co-ethylene glycol), poly(sebacic acid-co-1,4-butanediol), and poly(sebacic acid-co-lactic acid). Preferably, the sebacic acid copolymer is capryloyl glycerin/sebacic acid copolymer, hydrogenated castor oil/sebacic acid copolymer, or a combination thereof. In a preferred embodiment, the hair treatment composition includes capryloyl glycerin/sebacic acid copolymer, hydrogenated castor oil/sebacic acid copolymer, and combinations thereof. More preferably, the hair treatment composition includes hydrogenated castor oil/sebacic acid copolymer.

Hydrogenated castor oil/sebacic acid copolymer is a polymer created by reacting hydrogenated castor oil with sebacic acid. Castor oil is derived from the castor bean and contains ricinoleic acid (a fatty acid). Hydrogenation of castor oil involves the addition of hydrogen to the oil, converting its unsaturated fatty acids into saturated ones. This makes the oil more stable and solid at room temperature. Sebacic acid is a dicarboxylic acid typically derived from castor oil, sebacic acid has a long aliphatic chain and is often used to make esters or polymerize with other molecules.

The total amount of the one or more sebacic acid copolymers in the hair treatment composition will vary but is typically from about 0.1 to about 5 wt. %, based on the total weight of the hair treatment composition. In further embodiments, the hair treatment composition includes from about 0.1 to about 4 wt. %, about 0.1 to about 3 wt. %, about 0.1 to about 2 wt. %, about 0.1 to about 1 wt. %, about 0.3 to about 5 wt. %, about 0.3 to about 4 wt. %, about 0.3 to about 3 wt. %, about 0.3 to about 2 wt. %, about 0.3 to about 1.5 wt. %, about 0.3 to about 1 wt. %, about 0.5 to about 5 wt. %, about 0.5 to about 4 wt. %, about 0.5 to about 3 wt. %, about 0.5 to about 2 wt. %, about 0.5 to about 1.5 wt. %, or about 0.5 to about 1 wt. % of the one or more sebacic acid copolymers, based on the total weight of the hair treatment composition.

Preferably, the hair treatment composition includes from about 0.1 to about 5 wt. %, more preferably from about 0.2 to about 4 wt. %, and more preferably from about 0.3 to about 3 wt. %, and even more preferably from about 0.4 to about 2 wt. % of the one or more sebacic acid copolymers, based on the total weight of the hair treatment composition.

### (c) Vegetable Oils

Vegetable oils are natural oils extracted from the seeds, nuts, or fruits of plants. They are primarily composed of triglycerides, which consist of glycerol and fatty acids (saturated, monounsaturated, and polyunsaturated). They are typically rich in essential fatty acids such as oleic acid, linoleic acid, and linolenic acid, and often provide a good source of vitamins and antioxidants.

Nonlimiting examples of vegetable oils include castor oil, arachis oil, amaranth seed oil, apricot oil, argan oil, avocado oil, babassu oil, cottonseed oil, borage seed oil, camelina oil, camellia oil, safflower oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, manila oil, evening primrose oil, olive oil, palm oil, palm kernel oil, brazil nut oil, pecan nut oil, peach kernel oil, rapeseed oil, rosehip oil, sea buckthorn fruit oil, sea buckthorn kernel oil, sesame oil, soybean oil, sunflower oil, grapeseed oil, walnut oil, wild rose oil, wheat germ oil, coconut oil, avocado oil, arachis oil, apricot oil, argan oil, and babassu oil.

Additional, less common but nonlimiting examples of vegetable oils include baobab oil, black cumin seed oil, broccoli seed oil, buriti oil, cacay oil, chia seed oil, kalahari melon seed oil, kukui nut oil, maracuja oil, moringa oil, oat oil, papaya seed oil, plum kernel oil, prickly pear seed oil, raspberry seed oil, tamanu oil, tomato seed oil, and watermelon seed oil.

Preferably, the one or more vegetable oils comprises or consists of castor oil.

The total amount of the one or more vegetable oils in the hair treatment composition will vary but is typically from about 0.1 to about 10 wt. %, based on the total weight of the hair treatment composition. In further embodiments, the hair treatment composition includes from about 0.1 to about 8 wt. %, about 0.1 to about 5 wt. %, about 0.1 to about 3 wt. %, about 0.1 to about 2 wt. %, about 0.2 to about 10 wt. %, about 0.2 to about 8 wt. %, about 0.2 to about 5 wt. %, about 0.2 to about 3 wt. %, about 0.2 to about 2 wt. %, about 0.4 to about 10 wt. %, about 0.4 to about 8 wt. %, about 0.4 to about 5 wt. %, about 0.4 to about 3 wt. %, or about 0.4 to about 2 wt. % of the one or more vegetable oils, based on the total weight of the hair treatment composition.

Preferably, the hair treatment composition includes from about 0.1 to about 6 wt. %, preferably from about 0.2 to about 5 wt. %, more preferably about 0.3 to about 4 wt. %, and even more preferably about 0.4 to about 3 wt. % of the one or more vegetable oils, based on the total weight of the hair treatment composition.

### (d) Fatty Acids

A fatty acid is a carboxylic acid with an aliphatic chain, for example, of 6 to 30 carbon atoms, preferably 8 to 28 carbon atoms, more preferably from 12 to 26 carbon atoms, which is either saturated or unsaturated, and branched or unbranched. Most naturally occurring fatty acids have an unbranched chain of an even number of carbon atoms, from 6 to 28. In some instances, naturally occurring fatty acids are preferred.

Nonlimiting examples of fatty acids include caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, isostearic acid, and a combination thereof.

In a preferred embodiment, the one or more fatty acids are selected from unsaturated fatty acids, preferably mono-unsaturated fatty acids. Unsaturated fatty acid carbon chains contain one or more double bonds with a terminal carboxylic group (—COOH). A fatty acid with a single double bond is termed “monounsaturated fatty acid,” and fatty acids with more than one double bond are termed “polyunsaturated fatty acids.” Nonlimiting examples of unsaturated fatty acids include myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, and a combination thereof. In a preferred embodiment, the one or more fatty acids includes oleic acid, lauric acid, or a combination thereof.

In a preferred embodiment, the one or more fatty acids are selected from non-linear fatty acids. The term “non-linear fatty acids” as used in the instant disclosure refers to unsaturated fatty acid and/or branched fatty acids. Unsaturated fatty acid carbon chains contain one or more double bonds with a terminal carboxylic group (—COOH). A fatty acid with a single double bond is termed “monounsaturated fatty acid,” and fatty acids with more than one double bond are termed “polyunsaturated fatty acids.” Nonlimiting examples of unsaturated fatty acids include myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, and a combination thereof. In a preferred embodiment, the one or more fatty acids includes oleic acid, and optionally one or more additional fatty acids.

Nonlimiting examples of branched fatty acids include isostearic acid, isolauric acid, isomyristic acid, isopalmitic acid, and a combination there.

In a particularly preferred embodiment, the one or more fatty acids are selected from stearic acid, palmitic acid, myristic acid, lauric acid, oleic acid, linoelic acid, and arachidonic acid; and even more preferably, the one or more fatty acid are selected from oleic acid and lauric acid.

The total amount of the one or more fatty acids in the hair treatment composition will vary but is typically from about 0.1 to about 10 wt. %, based on the total weight of the hair treatment composition. In further embodiments, the hair treatment composition includes from about 0.1 to about 8 wt. %, about 0.1 to about 5 wt. %, about 0.1 to about 3 wt. %, about 0.1 to about 1 wt. %, about 0.2 to about 10 wt. %, about 0.2 to about 8 wt. %, about 0.2 to about 5 wt. %, about 0.2 to about 3 wt. %, about 0.2 to about 1 wt. %, about 0.5 to about 10 wt. %, about 0.5 to about 8 wt. %, about 0.5 to about 5 wt. %, about 0.5 to about 3 wt. %, about 0.5 to about 1.5 wt. %, about 0.8 to about 10 wt. %, about 0.8 to about 8 wt. %, about 0.8 to about 5 wt. %, or about 0.8 to about 3 wt. %, about 0.8 to about 1.5 wt. % of the one or more fatty acids, based on the total weight of the hair treatment composition.

Preferably, the hair treatment composition includes from about 0.1 to about 8 wt. %, preferably from about 0.2 to about 5 wt. %, more preferably from about 0.3 to about 4 wt. %, and even more preferably about 0.5 to about 3 wt. % of the one or more fatty acids, based on the total weight of the hair treatment composition.

### Total Amount of (a), (b), (c), and (d)

The total amount of the one or more cationic amidoamine surfactants (a), the hydrogenated castor oil/sebacic acid copolymer (b), the one or more vegetable oils (c), and the one or more fatty acids (d) will vary, but is typically from about 2 to about 10 wt. %, based on the total weight of the hair treatment composition. In further embodiments, the total amount of (a), (b), (c), and (d) is from about 2 to about 8 wt. %, about 2 to about 6 wt. %, about 2 to about 5 wt. %, about 2.5 to about 10 wt. %, about 2.5 to about 8 wt. %, about 2.5 to about 6 wt. %, about 2.5 to about 5 wt. %, about 3 to about 10 wt. %, about 3 to about 8 wt. %, about 3 to about 6 wt. %, or about 3 to about 5 wt. %, based on the total weight of the hair treatment composition.

### (e) Fatty Alcohols

The term “fatty alcohol” means an alcohol comprising at least one hydroxyl group (OH), and typically comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In various embodiments, the hair treatment composition includes at least one solid fatty alcohol. Solid fatty alcohols are fatty alcohols that are solid at ambient temperature and at atmospheric pressure (25° C., 780 mmHg), and are insoluble in water, i.e., they have a water solubility of less than 1% by weight, preferably less than 0.5% by weight, at 25° C., 1 atm. The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms. Nonlimiting examples include lauryl alcohol (1-dodecanol); myristyl alcohol (1-tetradecanol), cetyl alcohol (1-hexadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), myricylic alcohol (1-triacontanol), and combinations thereof. In a preferred embodiment, the compositions include at least one solid fatty alcohol selected from myristyl alcohol, cetyl alcohol, stearyl alcohol, behenyl alcohol and combinations thereof such as cetylstearyl or cetearyl alcohol.

In various embodiments, the hair treatment composition includes at least one liquid fatty alcohol, in particular containing C10-C34 and preferably have branched carbon chains and/or have one or more, preferably 1 to 3 double bonds. They are preferably branched and/or unsaturated (C═C double bond) and contain from 12 to 40 carbon atoms. The liquid fatty alcohols may be represented by: R—OH, wherein R denotes a C12-C24 branched or straight alkyl group or an alkenyl group, R being optionally substituted by one or more hydroxy groups. In certain embodiments, the liquid fatty alcohols are selected from branched saturated alcohols. Preferably, R does not contain a hydroxyl group. Nonlimiting examples include oleyl alcohol, linoleyl alcohol, linolenyl alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and combinations thereof. In other embodiments, the compositions are free or essentially free from liquid fatty alcohols, including the liquid fatty alcohols referenced above.

In a preferred embodiment, the one or more fatty alcohols are linear (straight chain) saturated fatty alcohols with 10 to 30 carbon atoms, preferably from 12 to 28 carbon atoms, more preferably from 14 to 24 carbon atoms. Nonlimiting examples include decyl alcohol, undecyl alcohol, dodecyl alcohol, myristyl alcohol, cetyl alcohol, stearyl alcohol, cetearyl alcohol, behenyl alcohol, myricyl alcohol and combinations thereof.

The total amount of the one or more fatty alcohols in the hair treatment compositions will vary but is typically from about 0.1 to about 10 wt. %, based on the total weight of the hair treatment composition. In further embodiments, the hair treatment composition includes from about 0.1 to about 8 wt. %, about 0.1 to about 6 wt. %, about 0.5 to about 10 wt. %, about 0.5 to about 8 wt. %, about 0.5 to about 6 wt. %, about 1 to about 10 wt. %, about 1 to about 8 wt. %, about 1 to about 6 wt. %, about 2 to about 10 wt. %, about 2 to about 8 wt. %, about 2 to about 6 wt. %, about 3 to about 10 wt. %, about 3 to about 8 wt. %, about 3 to about 6 wt. %, about 4 to about 10 wt. %, about 4 to about 8 wt. %, or about 4 to about 6 wt. %, based on the total weight of the hair treatment composition. Preferably, the hair treatment composition includes from about 1 to about 10 wt. %, more preferably about 2 to about 8 wt. %, and even more preferably about 3 to about 7 wt. % of one or more fatty alcohols, based on the total weight of the hair treatment composition.

### Total Amount of (a), (b), (c), (d), and (e)

The total amount of the one or more cationic amidoamine surfactants (a), the hydrogenated castor oil/sebacic acid copolymer (b), the one or more vegetable oils (c), the one or more fatty acids (d), and the one or more fatty alcohols (e) will vary, but is typically from about 3 to about 15 wt. %, based on the total weight of the hair treatment composition. In further embodiments, the total amount of (a), (b), (c), and (d) is from about 3 to about 12 wt. %, about 3 to about 10 wt. %, about 4 to about 15 wt. %, about 5 to about 12 wt. %, about 4 to about 10 wt. %, about 4 to about 8 wt. %, about 4.5 to about 15 wt. %, about 4.5 to about 12 wt. %, about 4.5 to about 10 wt. %, or about 4.5 to about 8 wt. %, based on the total weight of the hair treatment composition.

(f) Polyol with 2 to 8 Carbon Atoms

A polyol with 2 to 8 carbon atoms is an organic compound containing multiple hydroxyl (—OH) groups and a carbon backbone ranging from 1 to 8 carbon atoms. Nonlimiting examples include glycerol, ethylene glycol, diethylene glycol, triethylene glycol, butylene glycol, propylene glycol, isoprene glycol, dipropylene glycol, hexylene glycol, 1,2-propanediol (propylene glycol), and 1,3-propanediol (propanediol).

In a preferred embodiment, at least one of the one or more polyols with 2 to 8 carbon atoms is propanediol (1,3-propanediol). In a further preferred embodiment, the hair treatment composition includes two or more polyols with 2 to 8 carbon atoms, wherein at least one of the two or more polyols is propanediol (1,3-propanediol). In a further preferred embodiment, the hair treatment composition includes two or more polyols with 2 to 8 carbon atoms, wherein at least one of the two or more polyols is glycerin. Preferably, the two or more polyols with 2 to 8 carbon atoms include at least propanediol (1,3-propanediol) and glycerol.

The total amount of the one or more polyols with 2 to 8 carbon atoms in the hair treatment composition will vary but is typically from about 5 to about 25 wt. %, based on the total weight of the hair treatment composition. In further embodiments, the hair treatment composition includes from about 5 to about 20 wt. %, about 5 to about 18 wt. %, about 5 to about 16 wt. %, about 8 to about 25 wt. %, about 8 to about 20 wt. %, about 8 to about 18 wt. %, about 8 to about 16 wt. %, about 10 to about 25 wt. %, about 10 to about 20 wt. %, about 10 to about 18 wt. %, or about 10 to about 16 wt. % of the one or more polyols with 2 to 8 carbon atoms, based on the total weight of the hair treatment composition.

Preferably, the hair treatment composition includes from about 5 to about 25 wt. %, more preferably about 6 to about 20 wt. %, and even more preferably about 8 to about 18 wt. % of the one or more polyol with 2 to 8 carbon atoms, based on the total weight of the hair treatment composition.

In a preferred embodiment, at least one of the one or more polyols with 2 to 8 carbon atoms is propanediol (1,3-propanediol). The amount of the propanediol in the hair treatment composition will vary but is typically from about 5 to about 25 wt. %, based on the total weight of the hair treatment composition. In further embodiments, the hair treatment composition includes from about 5 to about 20 wt. %, about 5 to about 18 wt. %, about 5 to about 16 wt. %, about 8 to about 25 wt. %, about 8 to about 20 wt. %, about 8 to about 18 wt. %, about 8 to about 16 wt. %, about 10 to about 25 wt. %, about 10 to about 20 wt. %, about 10 to about 18 wt. %, or about 10 to about 16 wt. % of the propanediol, based on the total weight of the hair treatment composition.

Preferably, the hair treatment composition includes from about 5 to about 25 wt. %, more preferably about 6 to about 20 wt. %, and even more preferably about 8 to about 18 wt. %, and even more preferably about 10 to about 15 wt. % of propanediol, based on the total weight of the hair treatment composition.

In a preferred embodiment, the hair treatment composition includes glycerin as one of the one or more polyols with 2 to 8 carbon atoms. The total amount of the glycerin in the hair treatment composition will vary but is typically from about 0.1 to about 10 wt. %, based on the total weight of the hair treatment composition.

In further embodiments, the hair treatment composition includes form about 0.1 to about 8 wt. %, about 0.1 to about 5 wt. %, about 0.1 to about 3 wt. %, about 0.2 to about 10 wt. %, about 0.2 to about 8 wt. %, about 0.2 to about 5 wt. %, about 0.2 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 4 wt. %, or about 1 to about 3 wt. % of the glycerin, based on the total weight of the hair treatment composition.

### (g) Cationic Polymers

The hair treatment composition optionally includes one or more cationic polymers. These polymers carry a positive charge (cationic), which allows them to bind to the negatively charged surfaces of the skin and hair. The cationic polymers can comprise mixtures of monomer units derived from amine- and/or quaternary ammonium-substituted monomer and/or compatible spacer monomers. Cationic polymers often provide conditioning benefits to the hair treatment compositions and therefore may be referred to as “cationic conditioning polymers.” Non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride.

The hair treatment composition may include or be chosen from polyquaterniums. For example, the hair treatment composition may include Polyquaternium-1 (ethanol, 2,2′,2″-nitrilotris-, polymer with 1,4-dichloro-2-butene and N,N,N′,N′-tetramethyl-2-butene-1,4-diamine), Polyquaternium-2, (poly[bis(2-chloroethyl) ether-alt-1,3-bis[3-(dimethylamino) propyl]urea]), Polyquaternium-4, (hydroxyethyl cellulose dimethyl diallylammonium chloride copolymer; Diallyldimethylammonium chloride-hydroxyethyl cellulose copolymer), Polyquaternium-5 (copolymer of acrylamide and quaternized dimethylammoniumethyl methacrylate), Polyquaternium-6 (poly(diallyldimethylammonium chloride)), Polyquaternium-7 (copolymer of acrylamide and diallyldimethylammonium chloride), Polyquaternium-8 (copolymer of methyl and stearyl dimethylaminoethyl ester of methacrylic acid, quaternized with dimethylsulphate), Polyquaternium-9 (homopolymer of N,N-(dimethylamino)ethyl ester of methacrylic acid, quaternized with bromomethane), Polyquaternium-10 (quaternized hydroxyethyl cellulose), Polyquaternium-11 (copolymer of vinylpyrrolidone and quaternized dimethylaminoethyl methacrylate), Polyquaternium-12 (ethyl methacrylate/abietyl methacrylate/diethylaminoethyl methacrylate copolymer quaternized with dimethyl sulfate), Polyquaternium-13 (ethyl methacrylate/oleyl methacrylate/diethylaminoethyl methacrylate copolymer quaternized with dimethyl sulfate), Polyquaternium-14 (trimethylaminoethylmethacrylate homopolymer), Polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), Polyquaternium-19 (copolymer of polyvinyl alcohol and 2,3-epoxypropylamine), Polyquaternium-20 (copolymer of polyvinyl octadecyl ether and 2,3-epoxypropylamine), Polyquaternium-22 (copolymer of acrylic acid and diallyldimethylammonium chloride), Polyquaternium-24 (auaternary ammonium salt of hydroxyethyl cellulose reacted with a lauryl dimethyl ammonium substituted epoxide), Polyquaternium-27 (block copolymer of Polyquaternium-2 and Polyquaternium-17), Polyquaternium-28 (copolymer of vinylpyrrolidone and methacrylamidopropyl trimethylammonium), Polyquaternium-29 (chitosan modified with propylen oxide and quaternized with epichlorhydrin), Polyquaternium-30 (ethanaminium, N-(carboxymethyl)-N, N-dimethyl-2-[(2-methyl-1-oxo-2-propen-1-yl)oxy]-, inner salt, polymer with methyl 2-methyl-2-propenoate), Polyquaternium-31 (N,N-dimethylaminopropyl-N-acrylamidine quatemized with diethylsulfate bound to a block of polyacrylonitrile), Polyquaternium-32 (poly(acrylamide 2-methacryloxyethyltrimethyl ammonium chloride)), Polyquaternium-33 (copolymer of trimethylaminoethylacrylate salt and acrylamide), Polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), Polyquaternium-36 (copolymer of N,N-dimethylaminoethylmethacrylate and buthylmethacrylate, quaternized with dimethylsulphate), Polyquaternium-37 (poly(2-methacryloxyethyltrimethylammonium chloride)), Polyquaternium-39 (terpolymer of acrylic acid, acrylamide and diallyldimethylammonium Chloride), Polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), Polyquaternium-44 (3-Methyl-1-vinylimidazolium methyl sulfate-N-vinylpyrrolidone copolymer), Polyquaternium-45 (copolymer of (N-methyl-N-ethoxyglycine)methacrylate and N,N-dimethylaminoethylmethacrylate, quaternized with dimethyl sulphate), Polyquaternium-46 (terpolymer of vinylcaprolactam, vinylpyrrolidone, and quaternized vinylimidazole), Polyquaternium-47 (terpolymer of acrylic acid, methacrylamidopropyl trimethylammonium chloride, and methyl acrylate), and/or Polyquaternium-67.

In some instances, the hair treatment composition includes one or more cationic polymers selected from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

The cationic polymers may be a polyquaternium. In certain embodiments, the cationic surfactants may be polyquaterniums selected from polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

In some embodiments, the one or more cationic polymers are chosen from cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, and mixtures thereof.

Nonlimiting examples of preferred cationic polymers include cationic polysaccharides or cationic polysaccharide derivatives (cationic polysaccharide compounds), cationic gum derivatives (cationic gum compounds), polyquaterniums, polymer derivatives of diallyldimethyl ammonium chloride, polymer derivatives of methacrylamidopropyltrimethylammonium chloride, cationic cellulose derivatives (cationic cellulose compounds), quaternized hydroxyethyl cellulose, cationic starch derivatives (cationic starch compounds), cationic guar gum derivatives (cationic guar compounds), copolymers of acrylamide and dimethyldiallyammonium chloride, and a mixture thereof.

In a preferred embodiment, the hair treatment composition includes one or more cationic polysaccharides. In the context of the instant disclosure cationic polysaccharide include cationic polysaccharide derivatives (e.g., derivatized to be cationic), for example, resulting in cationic cellulose (cellulose derivatized to be cationic), cationic starch (derivatized to be cationic), cationic guar (guar derivatized to be cationic). Nonlimiting examples of cationic polysaccharides include cationic celluloses, cationic starches, cationic dextrose, and cationic guars.

The term “cationic celluloses” specifically refers to “cationic cellulose derivatives,” meaning cellulose that has undergone cationic modification. A cationic celluse derivative is a chemically modified cellulose that carries positively charged (cationic) functional groups. Nonlimiting examples of cationic celluloses include polyquaternium-10, polyquaternium-67, hydroxyethylcellulose hydroxypropyltrimonium chloride, and cationic hydroxyethyl cellulose.

The term “cationic starches” specifically refers to “cationic starch derivatives,” meaming starch that has undergone cationic modification. A cationic starch derivative is a chemically modified starch that carries positively charged (cationic) functional groups Nonlimiting examples of cationic starches include hydroxypropyl starch phosphate, starch hydroxypropyltrimonium chloride, starch trimethylammonium chloride, hydroxypropyl oxidized starch PG trimonium chloride, and hydroxyethyl starch quaternary ammonium derivatives.

The term “cationic dextrose” specifically refers to “cationic dextrose derivatives.” A cationic dextrose derivative is a chemically modified dextrose that carries positively charged (cationic) functional groups. Nonlimiting examples include dextrose hydroxypropyltrimonium chloride, cationic maltodextrin, dextrose quaternary ammonium derivatives, hydroxyethyl dextrose ammonium compounds, and dextrose polymethacrylate derivatives.

The term “cationic guars” specifically refers to “cationic guar derivatives.” A cationic guar derivative is a chemically modified guar gum that carries positively charged (cationic) functional groups. Nonlimiting examples of cationic guars include guar hydroxypropyltrimonium chloride, hydroxypropyl guar hydroxypropyltrimonium chloride, guar dimethylhydroxypropyltrimonium chloride, guar polyquaternium-7, and guar hydroxypropyltrimonium methylsulfate.

In a preferred embodiment, the hair treatment composition includes one or more cationic guars selected from guar hydroxypropyltrimonium chloride, hydroxypropyl guar hydroxypropyltrimonium chloride, guar dimethylhydroxypropyltrimonium chloride, and guar hydroxypropyltrimonium methylsulfate.

The total amount of the one or more cationic polymers in the hair treatment composition, if present, will vary but is typically from about 0.02 to about 5 wt. %, based on the total weight of the hair treatment composition. In further embodiments, the hair treatment composition includes from about 0.02 to about 4 wt. %, about 0.02 to about 3 wt. %, about 0.02 to about 2 wt. %, about 0.02 to about 1 wt. %, about 0.02 to about 0.5 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.05 to about 0.5 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.2 to about 5 wt. %, about 0.2 to about 4 wt. %, about 0.2 to about 3 wt. %, about 0.2 to about 2 wt. %, or about 0.2 to about 1 wt. % of the one or more cationic polymers, based on the total weight of the hair treatment composition.

Preferably, the hair treatment composition includes from about 0.05 to about 4 wt. %, more preferably about 0.1 to about 3 wt. %, and even more preferably about 0.1 to about 2 wt. % of the one or more cationic polymers, based on the total weight of the hair treatment composition.

### (h) Water

The hair treatment composition includes an appreciable amount of water. The total amount of water will vary but is typically from about 50 to about 90 wt. %, based on the total weight of the hair treatment composition. In further embodiments, the total amount of water in the hair treatment composition is from about 50 to about 85 wt. %, about 50 to about 82 wt. %, about 60 to about 90 wt. %, about 60 to about 85 wt. %, about 60 to about 82 wt. %, about 65 to about 90 wt. %, about 65 to about 85 wt. %, about 65 to about 82 wt. %, about 70 to about 90 wt. %, about 70 to about 85 wt. %, about 70 to about 82 wt. %, about 75 to about 90 wt. %, about 75 to about 85 wt. %, or about 75 to about 82 wt. %, based on the total weight of the hair treatment composition.

### (i) Miscellaneous Ingredients

The hair treatment composition optionally includes (or optionally excludes) one or more miscellaneous ingredients. Miscellaneous ingredients are ingredients that are compatible with the hair treatment compositions and do not disrupt or materially affect the basic and novel properties of the compositions. Nonlimiting examples of miscellaneous ingredients include preservatives, fragrances, pH adjusters, salts, chelating agents, buffers, antioxidants, flavonoids, vitamins, botanical extracts, UV filtering agents, proteins, protein hydrolysates and/or isolates, fillers (e.g., organic and/or inorganic fillers such as talc, calcium carbonate, silica, etc.) composition colorants, etc.

In various embodiments, the hair treatment composition includes one or more miscellaneous ingredients selected from preservatives, fragrances, pH adjusters, salts, chelating agents, buffers, composition colorants, fillers (such as talc, calcium carbonate, silica, including hydrated silica), vitamins, botanical extracts, and a combination thereof.

In the context of the instant disclosure, a “composition colorant” is a compound that colors the composition but does not have an appreciable coloring effect on hair. A composition colorant is included to provide a color to the composition for aesthetic appeal but is not intended to impart coloring properties to hair. As an example, hair styling gels, can be found in a variety of different colors (e.g., light blue, light pink, etc.) yet application of the styling gel to hair does not visibly change the color of the hair.

The total amount of the one or more miscellaneous ingredients, if present, will vary but is typically from about 0.01 to about 10 wt. %, based on the total weight of the hair treatment composition. In further embodiments, the hair treatment composition includes from about 0.01 to about 8 wt. %, about 0.01 to about 5 wt. %, about 0.1 to about 10 wt. %, about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, or about 1 to about 5 wt. % of one or more miscellaneous ingredients, based on the total weight of the hair treatment composition. Preferably, the hair treatment composition includes from about 0.1 to about 10 wt. %, more preferably about 0.5 to about 8 wt. %, and even more preferably about 1 to about 5 wt. % of one or more miscellaneous ingredients, based on the total weight of the hair treatment composition.

### Nonionic and Anionic Polymers

The hair treatment composition optionally includes one or more nonionic polymers, anionic polymers, or combinations thereof. Nonionic polymers and anionic polymers differ primarily in their electrical charge. Anionic polymers carry a negative charge due to the presence of charged functional groups, such as carboxyl (—COO⁻) or sulfate (—SO₃⁻) groups, which can ionize in solution. Nonionic polymers, on the other hand, are electrically neutral, lacking any charged functional groups. They typically rely on non-electrostatic interactions like hydrogen bonding and van der Waals forces for their behavior in solution. In a preferred embodiment, the nonionic polymers and/or the anionic polymers are polysaccharides or polysaccharide-based polymers (derived from polysaccharides).

i. Nonionic Polymers

Examples of the nonionic polymer include polymers having a structural unit derived from a nonionic monomer. Examples of the nonionic monomers include (meth)acrylates having a hydrocarbon group derived from an aliphatic alcohol having 1 or more and 22 or less carbon atoms; N-vinyl-2-pyrrolidone; vinyl alcohol; polyalkylene glycol (meth)acrylates (n=1 to 30); alkoxypolyalkylene glycol mono(meth)acrylates (n=1 to 30); and (meth)acrylamides and derivatives thereof.

The nonionic polymer may further contain a structural unit derived from monomers other than the nonionic monomer. Examples of the other monomers include the aforementioned styrene-based monomers; the aforementioned aromatic group-containing (meth)acrylates; and vinyl acetate.

Nonlimiting examples of nonionic polymers include polyvinyl alcohol, polyvinyl acetal, polyurethane-polyurea, polyvinylpyrrolidone, copolymers of vinylpyrrolidone and other nonionic monomer, such as a vinylpyrrolidone/vinyl acetate copolymer, cellulose-based polymers, such as a hydroxyalkyl cellulose, polyethylene glycol, polypropylene glycol, polyglycerin, polyvinyl alcohol, pullulan, guar gum, poly-N,N-dimethylacrylamide, poly-N-vinylacetamide, poly-N-vinylformamide, and poly(2-alkyl-2-oxazoline). In the case where polyvinyl butyral is used as polyvinyl acetal, the acetalization degree of polyvinyl butyral is preferably 50 mol % or more, more preferably 55 mol % or more, still more preferably 60 mol % or more, and is preferably 80 mol % or less, more preferably 75 mol % or less, still more preferably 70 mol % or less.

Examples of commercially available nonionic polymers include polyvinyl butyral, such as S-LEC B Series (which are a trade name, manufactured by Sekisui Chemical Co., Ltd.); polyurethane polyurea, such as BAYCUSAN® Series (which are a trade name, manufactured by Covestro Japan Ltd.); hydroxyethyl cellulose, such as HEC DAICEL® SE900, HEC DAICEL® SE850, HEC DAICEL® SE600, HEC DAICEL® SE550, and HEC DAICEL® SE400 (all of which are a trade name, manufactured by Daicel FineChem Ltd.); highly polymerized polyethylene glycol, such as POLYOX® WSR N-12K, POLYOX® WSR N-60K, and POLYOX® WSR 301 (all of which are a trade name, manufactured by The Dow Chemical Company); PEO-27, PEO-18, PEO-15, and PEO-8 (all of which are a trade name of polyethylene oxide, manufactured by Sumitomo Seika Chemicals Co., Ltd.); polyvinylpyrrolidone, such as LUVISKOL® K90, LUVISKOL® K80, and LUVISKOL® K30 (all of which are a trade name, manufactured by BASF SE); and polyvinyl alcohol, such as GOHSENOL® Series (which are a trade name, manufactured by Mitsubishi Chemical Corporation).

In a preferred embodiment, the one or more nonionic polymers are selected from nonionic polysaccharides and modified nonionic polysaccharides (nonionic derivatives of polysaccharides). The modified nonionic polysaccharide may comprise hydroxyalkylations. In the context of the present application, the degree of hydroxyalkylation (molar substitution or MS) of the modified nonionic polysaccharides refers to the number of alkylene oxide molecules consumed by the number of free hydroxyl functions present on the polysaccharides. In one embodiment, the MS of the modified nonionic polysaccharide is in the range of 0 to 3. In another embodiment, the MS is in the range of 0.1 to 3. In still another embodiment, the MS is in the range of 0.1 to 2.

The nonionic polysaccharide may be selected from a variety of naturally derived or modified polysaccharides. Nonlimiting examples include pullulan, pullulan derivatives, inulin, guar, cellulose, konjac, agar, agarose, curdlan, hydroxypropylmethyl cellulose, hydroxyethyl cellulose, methyl cellulose, hydroxypropyl cellulose, hydroxypropyl guar, cellulose acetate, cellulose aldehyde, carboxymethyl starch, hydroxypropyl starch, hydroxyethyl starch, starch acetate, starch aldehyde, and combinations thereof.

Nonlimiting examples of cellulose and cellulose derivatives include hydroxyethylcellulose (HEC), hydroxypropylcellulose (HPC), methylcellulose (MC), ethylcellulose, hydroxypropyl methylcellulose (HPMC), carboxymethylcellulose (CMC), microcrystalline cellulose, nanocellulose (bacterial cellulose).

Nonlimiting examples of guar gum and its derivatives include guar gum, hydroxypropyl guar (HPG), guar hydroxypropyl ether, guar ethoxylate (PEG-Guar, polyethylene glycol guar), and methyl guar.

Nonlimiting examples of marine and algal polysaccharides include carrageenan (chondrus crispus extract, Irish moss extract), sodium alginate, potassium alginate, laminarin, fucogel (biosaccharide gum-1), and fucoidan.

Nonlimiting examples of plant-based gums and galactomannans include xanthan gum, sclerotium gum, tara gum (caesalpinia spinosa gum), konjac glucomannan, locust bean gum (carob gum), acacia senegal gum, and tragacanth gum.

Nonlimiting examples of fermentation-derived polysaccharides include biosaccharide gum-1 (fucogel), biosaccharide gum-2, dextran, pullulan, and levan.

Nonlimiting examples of starch and its derivatives include corn starch, tapioca starch, rice starch, potato starch, hydroxypropyl starch, hydroxypropyl starch phosphate, hydroxyethyl starch, acetylated starch, hydroxypropyl tapioca starch, dextrin, maltodextrin, octenylsuccinate starch, sodium starch octenylsuccinate, amylopectin, and pre-gelatinized starches.

Dextran is a polysaccharide that primarily comes from bacteria, particularly Leuconostoc and Streptococcus, through the fermentation of glucose. These bacteria synthesize dextran by linking glucose molecules together in long, branched chains via α-1,6-glycosidic bonds, with some branches connected by α-1,3-glycosidic bonds. Nonlimiting examples include dextran, hydroxypropyl dextran, methyl dextran, ethyl dextran, acetylated dextran, dextran hydroxyethyl ether, dextran isostearate, dextran laurate, dextran palmitate, dextran myristate, dextran behenate, dextran oleate, dextran stearate, hydroxyethyl dextran, hydroxybutyl dextran, hydroxypropylmethyl dextran, dextran polycaprolactone, dextran poly(lactic-co-glycolic acid) (dextran-plga), dextran-based polyethers, and dextran-based polyurethanes.

Nonlimiting examples of other natural polysaccharides include beta-glucan (e.g., from oats or yeast), pectin (from fruits), inulin (e.g., from chicory root or agave), fructan (e.g., from chicory root, artichoke, etc.), arabinogalactan (e.g., from larch tree), and lupin polysaccharides

Preferably, the one or more nonionic polymers are selected from sclerotium gum, polyglucose Linéaire, hydroxyethylcellulose (HEC), hydroxypropylcellulose (HPC), methylcellulose (MC), ethylcellulose, hydroxypropyl methylcellulose (HPMC), carboxymethylcellulose (CMC), microcrystalline cellulose, and nanocellulose (bacterial cellulose). Polyglucose linéaire is a type of polysaccharide composed of glucose molecules linked together in a single, unbranched chain.

The total amount of the one or more nonionic polymers in the hair treatment composition, if present, will vary but is typically from about 0.01 to about 5 wt. %, based on the total weight of the hair treatment composition. In further embodiments, the total amount of the one or more nonionic polymers is from about 0.01 to about 3 wt. %, about 0.01 to about 2 wt. %, about 0.05 to about 5 wt. %, about 0.05 to about 3 wt. %, about 0.05 to about 2 wt. %, about 0.1 to about 5 wt. %, about 0.1 to about 3 wt. %, or about 0.1 to about 2 wt. %, based on the total weight of the hair treatment composition.

ii. Anionic Polymers

The anionic polymer may be a polymer functionalized with at least one carboxylic acid moiety. Nonlimiting examples of anionic polymers include polyacrylate-15, polyacrylate-21, polyacrylate-17, polyacrylate-18, polyacrylate-19, a branched anionic acrylate copolymer polyacrylate-2 crosspolymer, acrylates crosspolymer-3, polyacrylate-14, and those sold under the SYNTRAN® series as commercially available from Interpolymer such as acrylates copolymer, styrene/acrylates/ammonium methacrylate copolymer, and ammonium acrylates copolymer, and anionic polyurethanes, such as polyurethane-1, polyurethane-34, polyurethane-14/AMP-acrylates copolymer blend, acrylates copolymers of two or more monomers of (meth)acrylic acid or one of their simple esters, or mixtures thereof.

Further nonlimiting examples of anionic polymers include polyacrylate-15, polyacrylate-21, polyacrylate-17, polyacrylate-18, polyacrylate-19, copolymers derived from crotonic acid, such as the vinyl acetate/crotonic acid copolymers and the vinyl acetate/crotonic acid copolymers grafted by polyethylene glycol, those anionic polymers sold under the fixate series, such as a branched block anionic polymer sold as FIXATE® g-100, those sold under the Carbopol series, such as acrylates crosspolymer-4 (Carbopol® aqua sf-2), acrylates crosspolymer-4 (Carbopol®aqua CC), acrylates copolymer (Carbopol®aqua sf-1) carbomers, acrylates/C10-30 alkyl acrylates crosspolymers, the polymer known under the inci name acrylates/steareth-20 methacrylate crosspolymer, acrylates/palmeth-25 acrylate copolymer, acrylates/palmeth-20 acrylate copolymer, or mixtures thereof.

In various embodiments, the one or more anionic polymers are selected from acrylates/palmeth-25 acrylate copolymer, acrylates/beheneth-25 methacrylate copolymer, acrylates/steareth-20 methacrylate copolymer, acrylates/steareth-20 itaconate copolymer, acrylates/ceteth-20 itaconate copolymer, acrylates/ceteth-20 methacrylate copolymer, acrylate/beheneth-25 itaconate copolymer, acrylate/palmeth-25 methacrylates copolymer, acrylate/steareth-50 acrylate copolymer, and acrylates/palmeth-25 itaconate copolymer.

While not exclusively a nonionic polymer, in various embodiments, the hair treatment composition may include one or more citrus peel powders. The one or more citrus peel powders may be used instead of the one or more nonionic polymers (in amounts set forth for the one or more nonionic polymers described throughout the disclosure) or may be used in combination with the one or more nonionic polymers (wherein the combinations are in amounts set forth for the one or more nonionic polymers described throughout the disclosure). For example, the hair treatment composition may include both the one or more nonionic polysaccharides and the one or more citrus peel powders. Nonlimiting examples of citrus peel powders include a citrus limon (lemon) peel powder and citrus aurantifolia (lime) peel powder. Citrus peel powder can be commercially purchased, as INCI name: Citrus Limon (Lemon) Peel Powder (and) Sclerotium Gum (and) Citrus Aurantifolia (Lime) Peel Powder. Thus, in a preferred embodiment, the hair treatment composition includes one or more citrus peel powders and one or more nonionic polysaccharides, such as sclerotium gum. Similarly, the hair treatment composition may include one or more citrus peel powders instead of the one or more nonionic polymers.

In a preferred embodiment, the one or more anionic polymers are selected form anionic polysaccharides or modified anionic polysaccharides. Nonlimiting examples of modified anionic polysaccharides include carboxymethyl cellulose (CMC), xanthan gum, sodium alginate, carrageenan, gellan gum, pectin, acacia gum, tamarind gum, tragacanth gum, sodium hyaluronate, alginic acid, galacturonic acid, chondroitin sulfate, glucuronic acid, polyglutamic acid, hyaluronic acid derivatives, polygalacturonic acid, sulfated chitin, sulfated fucan, sulfated galactan, sodium polyacrylate, and dextran sulfate.

The total amount of the one or more anionic polymers in the hair treatment composition, if present, will vary but is typically from about 0.01 to about 5 wt. %, based on the total weight of the hair treatment composition. In further embodiments, the total amount of the one or more anionic polymers is from about 0.01 to about 3 wt. %, about 0.01 to about 2 wt. %, about 0.05 to about 5 wt. %, about 0.05 to about 3 wt. %, about 0.05 to about 2 wt. %, about 0.1 to about 5 wt. %, about 0.1 to about 3 wt. %, or about 0.1 to about 2 wt. %, based on the total weight of the hair treatment composition.

Mono-Alcohols with 2 to 8 Carbon Atoms

The hair treatment composition optionally includes one or more mono-alcohols with 2 to 8 carbon atoms. Nonlimiting examples include ethanol, propanol, isopropanol, butanol, butan-2-ol, 2-methylpropan-1-ol (isobutanol), pentan-1-ol, pentan-2-ol, 2-methylbutan-1-ol, 3-methylbutan-1-ol (Isoamyl alcohol), hexan-1-ol, hexan-2-ol, heptan-1-ol, and octan-1-ol. The amount of mono-alcohols with 2 to 8 carbon atoms, if present, will vary but may be from about 0.01 to about 10 wt. %, based on the total weight of the hair treatment composition. In further embodiments, the hair treatment composition includes from about 0.01 to about 8 wt. %, about 0.01 to about 5 wt. %, about 0.01 to about 3 wt. %, about 0.1 to about 10 wt. %, about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, or about 1 to about 3 wt. % of the one or more mono-alcohols with 2 to 8 carbon atoms, based on the total weight of the hair treatment composition. Preferably, the hair treatment composition comprises from about 0.01 to about 8 wt. %, more preferably from about 0.1 to about 6 wt. %, and even more preferably from about 0.5 to about 5 wt. % of the one or more mono-alcohols with 2 to 8 carbon atoms.

In a preferred embodiment, the hair treatment composition is free or essentially free of mono-alcohols with 2 to 8 carbon atoms. For example, the hair treatment composition may be free or essentially free from ethanol. In preferred embodiments, the total amount of the one or more mono-alcohols with 2 to 8 carbon atoms is 5 wt. % or less, 4 wt. % or less, 3 wt. % or less, 2 wt. % or less, 1 wt. % or less, 0.5 wt. % or less, or 0.1 wt. % or less, based on the total weight of the hair treatment composition.

pH

The pH of the hair treatment composition will vary but is typically from about 2 to about 8. In further embodiments, the pH of the hair treatment compositions is from about 2 to about 7, about 2 to less than 7, about 2 to about 6, about 2 to about 5.5, about 2 to about 5, about 2 to about 4.5, about 2 to about 4, about 3 to about 8, about 3 to about 7, about 3 to less than 7, about 3 to about 6, about 3 to about 5.5, about 3 to about 5, about 3 to about 4.5, or about 3 to about 4.

Preferably, the pH of the hair treatment composition is from about 2 to about 7, more preferably about 2.5 to less than 7, even more preferably about 2.5 to about 6, and even more preferably about 3 to about 5.5.

### Viscosity

The viscosity of the hair treatment composition will vary but is typically from about 200 cP to about 50,000 cP at 25° C., for example, when measure using a Brookfield Ametek instrument, with RV-04 spindle at 10 RPM. In further embodiments, the viscosity of the hair treatment composition is from about 200 to about 40,000 cP, about 200 to about 30,000 cP, about 200 to about 25,000 cP, about 200 to about 20,000 cP, about 200 to about 10,000 cP, about 500 to about 50,000 cP, about 500 to about 40,000 cP, about 500 to about 30,000 cP, about 500 to about 25,000 cP, about 500 to about 20,000 cP, about 500 to about 10,000 cP, about 1,000 to about 50,000 cP, about 1,000 to about 40,000 cP, about 1,000 to about 30,000 cP, about 1,000 to about 25,000 cP, about 1,000 to about 20,000 cP, about 1,000 to about 10,000 cP, about 2,000 to about 50,000 cP, about 2,000 to about 40,000 cP, about 2,000 to about 30,000 cP, about 2,000 to about 25,000 cP, about 2,000 to about 20,000 cP, about 2,000 to about 10,000 cP, about 5,000 to about 50,000 cP, about 5,000 to about 40,000 cP, about 5,000 to about 30,000 cP, about 5,000 to about 25,000 cP, about 5,000 to about 20,000 cP, about 5,000 to about 10,000 cP at 25° C., for example, when measure using a Brookfield Ametek instrument, with RV-04 spindle at 10 RPM.

Preferably, the viscosity of the hair treatment composition is from 500 to about 30,000 cP, more preferably about 1,000 to about 25,000 cP, and even more preferably about 2,000 to about 20,000 cP at 25° C., for example, when measure using a Brookfield Ametek instrument, with RV-04 spindle at 10 RPM.

### Additional Embodiments

In various embodiments, the hair treatment composition optionally includes or excludes one or more components selected from polyethylene glycols (PEG), anionic surfactants, nonionic surfactants, anionic polymers, silicones, or combinations thereof. For example, the hair treatment composition may optionally include one or more polyethylene glycols, for example, those with at least 2, 3, 5, 10, or 100 repeating units. In other embodiments, the hair treatment composition is preferably free or essentially free from polyethylene glycols including those with at least 2, 3, 5, 10, or 100 repeating units. The amount of polyethylene glycols, if present is typically less than about 5 wt. %, based on the total weight of the hair treatment composition. More preferably, the polyethylene glycols are in an amount less an about 4 wt. %, less than about 3 wt. %, less than about 2 wt. %, less than about 1 wt. %, less than about 0.5 wt. %, or less than about 0.1 wt. %, based on the total weight of the hair treatment composition.

In various embodiments, the hair treatment composition optionally includes or excludes one or more anionic surfactants. Nonlimiting examples of anionic surfactants include sulfate surfactants, isethionate surfactants, sarcosinate surfactants, sulfonate surfactants, taurate surfactants, etc. The amount of anionic surfactants, if present is typically less than about 5 wt. %, based on the total weight of the hair treatment composition. Preferably, the anionic surfactants are in an amount less an about 4 wt. %, less than about 3 wt. %, less than about 2 wt. %, less than about 1 wt. %, less than about 0.5 wt. %, or less than about 0.1 wt. %, based on the total weight of the hair treatment composition.

In various embodiments, the hair treatment composition optionally includes or excludes one or more nonionic surfactants. A nonionic surfactant is a type of surfactant that does not carry an electrical charge in its hydrophilic (water-attracting) head group. These surfactants are characterized by their ability to stabilize formulations, enhance emulsification, and improve the solubility of ingredients without reacting with ions in the solution. The hydrophilic part of nonionic surfactants typically consists of functional groups like hydroxyl (—OH) or ethylene oxide chains. The amount of nonionic surfactants, if present is typically less than about 5 wt. %, based on the total weight of the hair treatment composition. Preferably, the nonionic surfactants are in an amount less an about 4 wt. %, less than about 3 wt. %, less than about 2 wt. %, less than about 1 wt. %, less than about 0.5 wt. %, or less than about 0.1 wt. %, based on the total weight of the hair treatment composition.

In various embodiments, the hair treatment composition optionally includes or excludes one or more anionic polymers. The term “anionic polymer” is understood as meaning those polymers which carry in a protic solvent under standard conditions at least one structural unit having permanently anionic groups, the anionic groups having to be compensated by counterions while maintaining electroneutrality. As contemplated herein, anionic groups are, for example, carboxylate, sulfate, or sulfonate groups. Nonlimiting examples of anionic polymers include anionic polyurethanes, sodium polynaphthalene sulfonate, sodium lignosulfonate, sodium carboxymethyl cellulose, sodium salt of hydrophobically modified maleic anhydride copolymer, sodium polyacrylate, sodium polymethacrylate, ammonium polyacrylate, ammonium polymethacrylate, sodium salt of polymethacrylic acid, polystyrene sulfonate salts, carrageenan salts, dextran sulfate salts, polyacrylic acid salts, poly(methacrylic acid) salts, alginic acid salts, carboxymethylcellulose salts, polystyrene sulfonate/polystyrene copolymer salts, polystyrene sulfonate/maleic acid copolymers salt, copolymers thereof and combinations thereof. The amount of anionic polymers, if present is typically less than about 5 wt. %, based on the total weight of the hair treatment composition. Preferably, the anionic polymers are in an amount less an about 4 wt. %, less than about 3 wt. %, less than about 2 wt. %, less than about 1 wt. %, less than about 0.5 wt. %, or less than about 0.1 wt. %, based on the total weight of the hair treatment composition.

In various embodiments, the hair treatment composition optionally includes or excludes one or more silicones. Nonlimiting examples of silicones include polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or 2-phenylethyl)trimethylsiloxysilicates. The amount of silicones, if present is typically less than about 5 wt. %, based on the total weight of the hair treatment composition. Preferably, the silicones are in an amount less an about 4 wt. %, less than about 3 wt. %, less than about 2 wt. %, less than about 1 wt. %, less than about 0.5 wt. %, or less than about 0.1 wt. %, based on the total weight of the hair treatment composition.

### Methods

The hair treatment compositions are useful for treating the hair, for example, for conditioning, managing, and improving the look and feel of hair. The compositions can be applied to wet or damp hair or diluted with water and then applied to hair. Upon application to the hair, the hair treatment composition may be massaged into the hair, for example, with the hands, and/or spread throughout the hair with a comb or brush. This results in a smoothing and softening of the hair, which reduces frizz, dryness, and unwanted volume. The compositions can remain on the hair indefinitely or can be rinsed from the hair prior to drying and styling of the hair. Preferably, the hair treatment compositions are rinsed from the hair prior to drying and styling the hair after being applied and massaged into the hair.

The hair treatment composition is unique in its ability to provide hair with improved manageability, long-lasting style and frizz control, and smoothness. Accordingly, the instant disclosure relates to methods for improving the manageability of hair, for imparting lasting style and frizz control, and for imparting smoothness. More specifically, the compositions may be used in methods for conditioning the hair, providing curl definition to the hair, providing frizz control to the hair, improving ease of combability and detangling of the hair, and providing smoothness to the hair.

### Kits

The hair treatment compositions may be incorporated into a kit. For example, the kits may include at least one hair treatment composition according to the instant disclosure and one or more additional compositions, for example, a shampoo, a conditioner, etc. The various compositions are separately contained in the kits. In some instances, the kits include one or more compositions according to the instant disclosure and a shampoo, both of which are separately contained. The kits may also include one or more compositions according to the instant disclosure, a shampoo, and a conditioner, all of which are separately contained. Instructions, mixing components, brushes, gloves, measuring tools, etc., may optionally be included in the kits.

The hair treatment compositions may be packaged in a variety of different containers, such as, for example, a ready-to-use container. Nonlimiting examples of useful packaging include tubes, jars, caps, unit dose packages, and bottles, including squeezable tubes and bottles. In various embodiments, the hair treatment composition of the instant disclosure are packaged as a spray product or included in a spray bottle, which allows a user to apply the compositions to the hair by spraying the composition onto the hair. The spray product or spray bottle may be a pump spray bottle that is manually actuated, or the spray bottle can be pressurized such that the composition is dispensed from a pressurized aerosol container. A propellant may be used to force the composition out of the container. Suitable propellants include, but are not limited to, a liquefiable gas or a halogenated propellant. Examples of suitable propellants include dimethyl ether and hydrocarbon propellants such as propane, n-butane, iso-butane, CFCs, and CFC-replacement propellants. The propellants may be used singly or admixed.

### PREFERRED EMBODIMENTS

In a preferred embodiment, the hair treatment composition comprises, consists essentially of, or consists of:
(a) about 0.1 to about 8 wt. %, preferably about 0.2 to about 6 wt. %, and more preferably about 0.5 to about 4 wt. %, and even more preferably about 0.5 to about 3 wt. % of one or more cationic amidoamine surfactants;
(b) about 0.1 to about 5 wt. %, preferably about 0.2 to about 4 wt. %, and more preferably about 0.3 to about 3 wt. %, and even more preferably about 0.5 to about 2 wt. % of one or more sebacic acid copolymers;
(c) about 0.1 to about 10 wt. %, preferably about 0.1 to about 5 wt. %, more preferably about 0.2 to about 3 wt. %, and even more preferably about 0.3 to about 2 wt. % of one or more vegetable oils;
(d) about 0.1 to about 10 wt. %, preferably about 0.5 to about 6 wt. %, more preferably about 0.8 to about 5 wt. %, and even more preferably about 1 to about 4 wt. % of one or more fatty acids;
   wherein preferably (a), (b), (c), and (d) are in a combined amounts of about 2 to about 10 wt. %, preferably about 2.5 to about 8 wt. %, more preferably about 3 to about 6 wt. %;
(e) optionally, about 0.01 to about 10 wt. %, preferably about 0.5 to about 5 wt. %, more preferably about 0.8 to about 5 wt. %, and even more preferably about 1 to about 3 wt. % of one or more fatty alcohols;
   wherein preferably (a), (b), (c), (d), and (e) are in a combined amount of about 3 to about 12 wt. %, preferably about 4 to about 10 wt. %, more preferably about 4.5 to about 8 wt. %;
(f) about 5 to about 25 wt. %, preferably about 6 to about 20 wt. %, more preferably about 8 to about 18 wt. %, and even more preferably about 12 to about 18 wt. % of one or more polyols with 2 to 8 carbon atoms;
(g) optionally, about 0.01 to about 5 wt. %, preferably about 0.05 to about 4 wt. %, more preferably about 0.1 to about 3 wt. % of one or more cationic polymers;
(h) about 60 to about 90 wt. %, preferably about 65 to about 85 wt. %, more preferably about 70 to about 85 wt. %, and even more preferably about 72 to about 84 wt. % of water;
(i) optionally, about 0.01 to about 10 wt. %, preferably about 0.05 to about 6 wt. %, more preferably about 0.1 to about 5 wt. % of one or more anionic polymers, one or more nonionic polymers, or combinations thereof; and
(j) optionally, about 0.01 to about 10 wt. %, preferably about 0.1 to about 8 wt. %, and more preferably about 0.5 to about 6 wt. %, and even more preferably about 1 to about 5 wt. % of one or more miscellaneous ingredients;
   wherein the hair treatment composition is an oil-in-water emulsion, and all percentages by weight are based on a total weight of the hair treatment composition.

In a preferred embodiment, the hair treatment composition comprises, consists essentially of, or consists of:
(a) about 0.1 to about 8 wt. %, preferably about 0.2 to about 6 wt. %, and more preferably about 0.5 to about 4 wt. %, and even more preferably about 0.5 to about 3 wt. % of one or more cationic amidoamine surfactants;
(b) about 0.1 to about 5 wt. %, preferably about 0.2 to about 4 wt. %, and more preferably about 0.3 to about 3 wt. %, and even more preferably about 0.5 to about 2 wt. % of capryloyl glycerin/sebacic acid copolymer, hydrogenated castor oil/sebacic acid copolymer, or a combination thereof, wherein preferably the hair treatment composition includes the hydrogenated castor oil/sebacic acid copolymer;
(c) about 0.1 to about 10 wt. %, preferably about 0.1 to about 5 wt. %, more preferably about 0.2 to about 3 wt. %, and even more preferably about 0.3 to about 2 wt. % of one or more vegetable oils, preferably castor oil;
(d) about 0.1 to about 10 wt. %, preferably about 0.5 to about 6 wt. %, more preferably about 0.8 to about 5 wt. %, and even more preferably about 1 to about 4 wt. % of one or more fatty acids;
   wherein preferably (a), (b), (c), and (d) are in a combined amounts of about 2 to about 10 wt. %, preferably about 2.5 to about 8 wt. %, more preferably about 3 to about 6 wt. %;
(e) optionally, about 0.01 to about 10 wt. %, preferably about 0.5 to about 5 wt. %, more preferably about 0.8 to about 5 wt. %, and even more preferably about 1 to about 3 wt. % of one or more fatty alcohols;
   wherein preferably (a), (b), (c), (d), and (e) are in a combined amount of about 3 to about 12 wt. %, preferably about 4 to about 10 wt. %, more preferably about 4.5 to about 8 wt. %;
(f) about 5 to about 25 wt. %, preferably about 6 to about 20 wt. %, more preferably about 8 to about 18 wt. %, and even more preferably about 12 to about 18 wt. % of one or more polyols with 2 to 8 carbon atoms;
(g) optionally, about 0.01 to about 5 wt. %, preferably about 0.05 to about 4 wt. %, more preferably about 0.1 to about 3 wt. % of one or more cationic polymers, preferably one or more cationic polysaccharides;
(h) about 60 to about 90 wt. %, preferably about 65 to about 85 wt. %, more preferably about 70 to about 85 wt. %, and even more preferably about 72 to about 84 wt. % of water;
(i) optionally, about 0.01 to about 10 wt. %, preferably about 0.05 to about 6 wt. %, more preferably about 0.1 to about 5 wt. % of one or more anionic polymers, one or more nonionic polymers, or combinations thereof; and
(j) optionally, about 0.01 to about 10 wt. %, preferably about 0.1 to about 8 wt. %, and more preferably about 0.5 to about 6 wt. %, and even more preferably about 1 to about 5 wt. % of one or more miscellaneous ingredients;
   wherein the hair treatment composition is an oil-in-water emulsion, and all percentages by weight are based on a total weight of the hair treatment composition.

In a preferred embodiment, the hair treatment composition comprises, consists essentially of, or consists of:
(a) about 0.1 to about 8 wt. %, preferably about 0.2 to about 6 wt. %, and more preferably about 0.5 to about 4 wt. %, and even more preferably about 0.5 to about 3 wt. % of one or more cationic amidoamine surfactants selected from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamidopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, and stearamidoethyldiethylamine;
(b) about 0.1 to about 5 wt. %, preferably about 0.2 to about 4 wt. %, and more preferably about 0.3 to about 3 wt. %, and even more preferably about 0.5 to about 2 wt. % of capryloyl glycerin/sebacic acid copolymer, hydrogenated castor oil/sebacic acid copolymer, or a combination thereof, wherein preferably the hair treatment composition includes the hydrogenated castor oil/sebacic acid copolymer;
(c) about 0.1 to about 10 wt. %, preferably about 0.1 to about 5 wt. %, more preferably about 0.2 to about 3 wt. %, and even more preferably about 0.3 to about 2 wt. % of one or more vegetable oils, preferably castor oil;
(d) about 0.1 to about 10 wt. %, preferably about 0.5 to about 6 wt. %, more preferably about 0.8 to about 5 wt. %, and even more preferably about 1 to about 4 wt. % of one or more fatty acids;
   wherein preferably (a), (b), (c), and (d) are in a combined amounts of about 2 to about 10 wt. %, preferably about 2.5 to about 8 wt. %, more preferably about 3 to about 6 wt. %;
(e) optionally, about 0.01 to about 10 wt. %, preferably about 0.5 to about 5 wt. %, more preferably about 0.8 to about 5 wt. %, and even more preferably about 1 to about 3 wt. % of one or more fatty alcohols;
   wherein preferably (a), (b), (c), (d), and (e) are in a combined amount of about 3 to about 12 wt. %, preferably about 4 to about 10 wt. %, more preferably about 4.5 to about 8 wt. %;
(f) about 5 to about 25 wt. %, preferably about 6 to about 20 wt. %, more preferably about 8 to about 18 wt. %, and even more preferably about 12 to about 18 wt. % of one or more polyols with 2 to 8 carbon atoms selected from ethylene glycol, propylene glycol, butylene glycol, hexylene glycol, pentylene glycol, 1,3-propanediol, diethylene glycol, dipropylene glycol, caprylyl glycol, and glycerin, preferably wherein the one or more polyols with 2 to 8 carbon atoms are selected from glycerin, 1,3-propanediol, and 1,2-propanediol;
(g) optionally, about 0.01 to about 5 wt. %, preferably about 0.05 to about 4 wt. %, more preferably about 0.1 to about 3 wt. % of one or more cationic polymers, preferably one or more cationic polysaccharides, more preferably wherein the one or more cationic polysaccharides are cationic guars selected from guar hydroxypropyltrimonium chloride, hydroxypropyl guar hydroxypropyltrimonium chloride, guar dimethylhydroxypropyltrimonium chloride, and guar hydroxypropyltrimonium methylsulfate;
(h) about 60 to about 90 wt. %, preferably about 65 to about 85 wt. %, more preferably about 70 to about 85 wt. %, and even more preferably about 72 to about 84 wt. % of water;
(i) optionally, about 0.01 to about 10 wt. %, preferably about 0.05 to about 6 wt. %, more preferably about 0.1 to about 5 wt. % of one or more anionic polymers, one or more nonionic polymers, or combinations thereof; and
(j) optionally, about 0.01 to about 10 wt. %, preferably about 0.1 to about 8 wt. %, and more preferably about 0.5 to about 6 wt. %, and even more preferably about 1 to about 5 wt. % of one or more miscellaneous ingredients, wherein the one or more miscellaneous ingredients are preferably selected from preservatives, fragrances, pH adjusters, salts, chelating agents, buffers, composition colorants, particulate fillers (such as talc, calcium carbonate, silica, including hydrated silica), vitamins, botanical extracts, and a combination thereof;
   wherein the hair treatment composition is an oil-in-water emulsion, and all percentages by weight are based on a total weight of the hair treatment composition.

In a preferred embodiment, the hair treatment composition comprises, consists essentially of, or consists of:
(a) about 0.1 to about 8 wt. %, preferably about 0.2 to about 6 wt. %, and more preferably about 0.5 to about 4 wt. %, and even more preferably about 0.5 to about 3 wt. % of one or more cationic amidoamine surfactants selected from stearamidopropyl dimethylamine, stearamidoethyl dimethylamine, brassicaamidopropyl dimethylamine, oleamidopropyl dimethylamine, and behenamidopropyl dimethylamine:
(b) about 0.1 to about 5 wt. %, preferably about 0.2 to about 4 wt. %, and more preferably about 0.3 to about 3 wt. %, and even more preferably about 0.5 to about 2 wt. % of hydrogenated castor oil/sebacic acid copolymer;
(c) about 0.1 to about 10 wt. %, preferably about 0.1 to about 5 wt. %, more preferably about 0.2 to about 3 wt. %, and even more preferably about 0.3 to about 2 wt. % of castor oil;
(d) about 0.1 to about 10 wt. %, preferably about 0.5 to about 6 wt. %, more preferably about 0.8 to about 5 wt. %, and even more preferably about 1 to about 4 wt. % of one or more fatty acids, wherein the one or more fatty acids are preferably selected from myristic acid, lauric acid, palmitic acid, stearic acid, behenic acid, arichidonic acid, oleic acid, isostearic acid, and sebacic acid;
   wherein preferably (a), (b), (c), and (d) are in a combined amounts of about 2 to about 10 wt. %, preferably about 2.5 to about 8 wt. %, more preferably about 3 to about 6 wt. %;
(e) optionally, about 0.01 to about 10 wt. %, preferably about 0.5 to about 5 wt. %, more preferably about 0.8 to about 5 wt. %, and even more preferably about 1 to about 3 wt. % of one or more fatty alcohols, wherein if present, the one or more fatty alcohols are preferably selected from octyldodecanol, decyl alcohol, undecyl alcohol, dodecyl alcohol, myristyl alcohol, cetearyl alcohol, cetyl alcohol, stearyl alcohol, isostearyl alcohol, isocetyl alcohol, behenyl alcohol, linalool, oleyl alcohol, isotridecyl alcohol, and myricyl alcohol;
   wherein preferably (a), (b), (c), (d), and (e) are in a combined amount of about 3 to about 12 wt. %, preferably about 4 to about 10 wt. %, more preferably about 4.5 to about 8 wt. %;
(f) about 5 to about 25 wt. %, preferably about 6 to about 20 wt. %, more preferably about 8 to about 18 wt. %, and even more preferably about 12 to about 18 wt. % of one or more polyols with 2 to 8 carbon atoms selected from ethylene glycol, propylene glycol, butylene glycol, hexylene glycol, pentylene glycol, 1,3-propanediol, diethylene glycol, dipropylene glycol, caprylyl glycol, and glycerin, preferably wherein the one or more polyols with 2 to 8 carbon atoms are selected from glycerin, 1,3-propanediol, and 1,2-propanediol, wherein even more preferably, the one or more polyols with 2 to 8 carbon atoms include or consist of both 1,3-propanediol and glycerin, wherein the 1,3-propanediol and glycerin are in a weight ratio of about 4:1 to about 8:1, preferably about 5:1 to about 8:1;
(g) optionally, about 0.01 to about 5 wt. %, preferably about 0.05 to about 4 wt. %, more preferably about 0.1 to about 3 wt. % of one or more cationic polymers, preferably one or more cationic polysaccharides, more preferably wherein the one or more cationic polysaccharides are cationic guars selected from guar hydroxypropyltrimonium chloride, hydroxypropyl guar hydroxypropyltrimonium chloride, guar dimethylhydroxypropyltrimonium chloride, and guar hydroxypropyltrimonium methylsulfate;
(h) about 60 to about 90 wt. %, preferably about 65 to about 85 wt. %, more preferably about 70 to about 85 wt. %, and even more preferably about 72 to about 84 wt. % of water;
(i) optionally, about 0.01 to about 10 wt. %, preferably about 0.05 to about 6 wt. %, more preferably about 0.1 to about 5 wt. % of one or more anionic polymers, one or more nonionic polymers, or combinations thereof, wherein preferably, the one or more anionic polymers, the one or more nonionic polymers, or the combination comprises or consists of anionic polysaccharides and nonionic polysaccharides; and
(j) optionally, about 0.01 to about 10 wt. %, preferably about 0.1 to about 8 wt. %, and more preferably about 0.5 to about 6 wt. %, and even more preferably about 1 to about 5 wt. % of one or more miscellaneous ingredients, wherein the one or more miscellaneous ingredients are preferably selected from preservatives, fragrances, pH adjusters, salts, chelating agents, buffers, composition colorants, particulate fillers (such as talc, calcium carbonate, silica, including hydrated silica), vitamins, botanical extracts, and a combination thereof;
   wherein the hair treatment composition is an oil-in-water emulsion, and all percentages by weight are based on a total weight of the hair treatment composition.

In a preferred embodiment, the hair treatment composition comprises, consists essentially of, or consists of:
(a) about 0.1 to about 8 wt. %, preferably about 0.2 to about 6 wt. %, and more preferably about 0.5 to about 4 wt. %, and even more preferably about 0.5 to about 3 wt. % of one or more cationic amidoamine surfactants selected from stearamidopropyl dimethylamine, stearamidoethyl dimethylamine, and brassicaamidopropyl dimethylamine, preferably the one or more cationic amidoamine surfactants are selected from stearamidopropyl dimethylamine and brassicaamidopropyl dimethylamine;
(b) about 0.1 to about 5 wt. %, preferably about 0.2 to about 4 wt. %, and more preferably about 0.3 to about 3 wt. %, and even more preferably about 0.5 to about 2 wt. % of hydrogenated castor oil/sebacic acid copolymer;
(c) about 0.1 to about 10 wt. %, preferably about 0.1 to about 5 wt. %, more preferably about 0.2 to about 3 wt. %, and even more preferably about 0.3 to about 2 wt. % of castor oil;
(d) about 0.1 to about 10 wt. %, preferably about 0.5 to about 6 wt. %, more preferably about 0.8 to about 5 wt. %, and even more preferably about 1 to about 4 wt. % of one or more fatty acids, wherein the one or more fatty acids are preferably selected from myristic acid, lauric acid, palmitic acid, stearic acid, behenic acid, arichidonic acid, oleic acid, isostearic acid, and sebacic acid;
   wherein preferably (a), (b), (c), and (d) are in a combined amounts of about 2 to about 10 wt. %, preferably about 2.5 to about 8 wt. %, more preferably about 3 to about 6 wt. %;
(e) optionally, about 0.01 to about 10 wt. %, preferably about 0.5 to about 5 wt. %, more preferably about 0.8 to about 5 wt. %, and even more preferably about 1 to about 3 wt. % of one or more fatty alcohols, wherein if present, the one or more fatty alcohols are preferably selected from octyldodecanol, decyl alcohol, undecyl alcohol, dodecyl alcohol, myristyl alcohol, cetearyl alcohol, cetyl alcohol, stearyl alcohol, isostearyl alcohol, isocetyl alcohol, behenyl alcohol, linalool, oleyl alcohol, isotridecyl alcohol, and myricyl alcohol;
   wherein preferably (a), (b), (c), (d), and (e) are in a combined amount of about 3 to about 12 wt. %, preferably about 4 to about 10 wt. %, more preferably about 4.5 to about 8 wt. %;
(f) about 5 to about 25 wt. %, preferably about 6 to about 20 wt. %, more preferably about 8 to about 18 wt. %, and even more preferably about 12 to about 18 wt. % of one or more polyols with 2 to 8 carbon atoms selected from ethylene glycol, propylene glycol, butylene glycol, hexylene glycol, pentylene glycol, 1,3-propanediol, diethylene glycol, dipropylene glycol, caprylyl glycol, and glycerin, preferably wherein the one or more polyols with 2 to 8 carbon atoms are selected from glycerin, 1,3-propanediol, and 1,2-propanediol, wherein even more preferably, the one or more polyols with 2 to 8 carbon atoms include or consist of both 1,3-propanediol and glycerin, wherein the 1,3-propanediol and glycerin are in a weight ratio of about 4:1 to about 8:1, preferably about 5:1 to about 8:1;
(g) optionally, about 0.01 to about 5 wt. %, preferably about 0.05 to about 4 wt. %, more preferably about 0.1 to about 3 wt. % of one or more cationic polymers, preferably one or more cationic polysaccharides, more preferably wherein the one or more cationic polysaccharides are cationic guars selected from guar hydroxypropyltrimonium chloride, hydroxypropyl guar hydroxypropyltrimonium chloride, guar dimethylhydroxypropyltrimonium chloride, and guar hydroxypropyltrimonium methylsulfate;
(h) about 60 to about 90 wt. %, preferably about 65 to about 85 wt. %, more preferably about 70 to about 85 wt. %, and even more preferably about 72 to about 84 wt. % of water;
(i) optionally, about 0.01 to about 10 wt. %, preferably about 0.05 to about 6 wt. %, more preferably about 0.1 to about 5 wt. % of one or more nonionic polymers, wherein preferably, the one or more nonionic polymers are selected from nonionic polysaccharides and the hair treatment composition is preferably free from anionic polymers; and
(j) optionally, about 0.01 to about 10 wt. %, preferably about 0.1 to about 8 wt. %, and more preferably about 0.5 to about 6 wt. %, and even more preferably about 1 to about 5 wt. % of one or more miscellaneous ingredients, wherein the one or more miscellaneous ingredients are preferably selected from preservatives, fragrances, pH adjusters, salts, chelating agents, buffers, composition colorants, particulate fillers (such as talc, calcium carbonate, silica, including hydrated silica), vitamins, botanical extracts, and a combination thereof;
   wherein the hair treatment composition is an oil-in-water emulsion, and all percentages by weight are based on a total weight of the hair treatment composition.

In a preferred embodiment, the hair treatment composition comprises, consists essentially of, or consists of:
(a) about 0.1 to about 8 wt. %, preferably about 0.2 to about 6 wt. %, and more preferably about 0.5 to about 4 wt. %, and even more preferably about 0.5 to about 3 wt. % of stearamidopropyl dimethylamine;
(b) about 0.1 to about 5 wt. %, preferably about 0.2 to about 4 wt. %, and more preferably about 0.3 to about 3 wt. %, and even more preferably about 0.5 to about 2 wt. % of hydrogenated castor oil/sebacic acid copolymer;
(c) about 0.1 to about 10 wt. %, preferably about 0.1 to about 5 wt. %, more preferably about 0.2 to about 3 wt. %, and even more preferably about 0.3 to about 2 wt. % of castor oil;
(d) about 0.1 to about 10 wt. %, preferably about 0.5 to about 6 wt. %, more preferably about 0.8 to about 5 wt. %, and even more preferably about 1 to about 4 wt. % of one or more fatty acids, wherein the one or more fatty acids are preferably selected from myristic acid, lauric acid, palmitic acid, stearic acid, behenic acid, arichidonic acid, oleic acid, isostearic acid, and sebacic acid;
   wherein preferably (a), (b), (c), and (d) are in a combined amounts of about 2 to about 10 wt. %, preferably about 2.5 to about 8 wt. %, more preferably about 3 to about 6 wt. %;
(e) optionally, about 0.01 to about 10 wt. %, preferably about 0.5 to about 5 wt. %, more preferably about 0.8 to about 5 wt. %, and even more preferably about 1 to about 3 wt. % of one or more fatty alcohols, wherein if present, the one or more fatty alcohols are preferably selected from octyldodecanol, decyl alcohol, undecyl alcohol, dodecyl alcohol, myristyl alcohol, cetearyl alcohol, cetyl alcohol, stearyl alcohol, isostearyl alcohol, isocetyl alcohol, behenyl alcohol, linalool, oleyl alcohol, isotridecyl alcohol, and myricyl alcohol;
   wherein preferably (a), (b), (c), (d), and (e) are in a combined amount of about 3 to about 12 wt. %, preferably about 4 to about 10 wt. %, more preferably about 4.5 to about 8 wt. %;
(f) about 5 to about 25 wt. %, preferably about 6 to about 20 wt. %, more preferably about 8 to about 18 wt. %, and even more preferably about 12 to about 18 wt. % of one or more polyols with 2 to 8 carbon atoms selected from ethylene glycol, propylene glycol, butylene glycol, hexylene glycol, pentylene glycol, 1,3-propanediol, diethylene glycol, dipropylene glycol, caprylyl glycol, and glycerin, preferably wherein the one or more polyols with 2 to 8 carbon atoms are selected from glycerin, 1,3-propanediol, and 1,2-propanediol, wherein even more preferably, the one or more polyols with 2 to 8 carbon atoms include or consist of both 1,3-propanediol and glycerin, wherein the 1,3-propanediol and glycerin are in a weight ratio of about 4:1 to about 8:1, preferably about 5:1 to about 8:1;
(g) optionally, about 0.01 to about 5 wt. %, preferably about 0.05 to about 4 wt. %, more preferably about 0.1 to about 3 wt. % of one or more cationic polymers, preferably one or more cationic polysaccharides, more preferably wherein the one or more cationic polysaccharides are cationic guars selected from guar hydroxypropyltrimonium chloride, hydroxypropyl guar hydroxypropyltrimonium chloride, guar dimethylhydroxypropyltrimonium chloride, and guar hydroxypropyltrimonium methylsulfate, wherein even more preferably the one or more cationic polymers comprises or consists of guar hydroxypropyltrimonium chloride;
(h) about 60 to about 90 wt. %, preferably about 65 to about 85 wt. %, more preferably about 70 to about 85 wt. %, and even more preferably about 72 to about 84 wt. % of water;
(i) optionally, about 0.01 to about 10 wt. %, preferably about 0.05 to about 6 wt. %, more preferably about 0.1 to about 5 wt. % of one or more nonionic polymers, wherein preferably, the one or more nonionic polymers are selected from nonionic polysaccharides and the hair treatment composition is preferably free from anionic polymers; and
(j) optionally, about 0.01 to about 10 wt. %, preferably about 0.1 to about 8 wt. %, and more preferably about 0.5 to about 6 wt. %, and even more preferably about 1 to about 5 wt. % of one or more miscellaneous ingredients, wherein the one or more miscellaneous ingredients are preferably selected from preservatives, fragrances, pH adjusters, salts, chelating agents, buffers, composition colorants, particulate fillers (such as talc, calcium carbonate, silica, including hydrated silica), vitamins, botanical extracts, and a combination thereof;
   wherein the hair treatment composition is an oil-in-water emulsion, and all percentages by weight are based on a total weight of the hair treatment composition.

### EXAMPLES

Various changes can be made in the above-described compositions and methods without departing from the scope of the invention. Accordingly, it is intended that all disclosure contained in the above description and in the examples given below, shall be interpreted as illustrative and not in a limiting sense.

### Example 1

### Compositions

TABLE I

| | | | Inventive | | Comparative | | | |
|---|---|---|---|---|---|---|---|---|
| | | | A1 | A | B | C | D | E |
| (a) | Cationic | STEARAMIDOPROPYL | 0.8 | 0.8 | 0.6 | 0.6 | 0.6 | 0.6 |
| | amidoamine | DIMETHYLAMINE | | | | | | |
| (b) | Polymer | HYDROGENATED CASTOR | 0.7 | 0.7 | 0.7 | 0.5 | 0.5 | 0.5 |
| | | OIL/SEBACIC ACID | | | | | | |
| | | COPOLYMER | | | | | | |
| (c) | Oil | CASTOR OIL | 0.4 | 0.4 | 0.4 | | 0.3 | 0.5 |
| (d) | Fatty Acid | OLEIC ACID | 0.8 | 0.8 | | | | |
| | | LAURIC ACID | 0.2 | 0.2 | | | | |
| Total amount of (a) + (b) + (c) + (d) | | | 2.9 | 2.9 | 1.7 | 1.1 | 1.4 | 1.1 |
| (e) | Optional Fatty | CETEARYL ALCOHOL | 1.9 | 1.9 | 1.3 | 1.3 | 1.3 | 1.3 |
| | Alcohol | OCTYLDODECANOL | | | | 0.5 | 0.3 | |
| Total amount of (a) + (b) + (c) + (d) + (e) | | | 4.8 | 4.8 | 3 | 2.9 | 3.0 | 2.4 |
| (f) | Polyol | PROPANEDIOL | 13 | 13 | 13 | 13 | 13 | 13 |
| | | GLYCERIN | 2 | 2 | 2 | 2 | 2 | 2 |
| (g) | Cationic | GUAR HYDROXYPROPYL- | | | 0.2 | 0.2 | 0.2 | 0.2 |
| | Polymer | TRIMONIUM CHLORIDE | | | | | | |
| (h) | Water | WATER | QS 100 | | | | | |
| | Optional Film | CANDELILLA WAX | | 0.2 | | | | |
| | Former | EXTRACT | | | | | | |
| (i) | Miscellaneous ¹ | LACTIC ACID | 0.2 | 0.2 | 0.2 | 0.2 | 0.2 | 0.2 |
| | (≤5 wt. %) | PHENOXYETHANOL | 0.8 | 0.8 | 0.8 | 0.8 | 0.8 | 0.8 |
| | | FRAGRANCE | 0.3 | 0.3 | 0.3 | 0.3 | 0.3 | 0.3 |

| | | | | | | |
|---|---|---|---|---|---|---|
| pH | 4 | 4 | 4 | 4 | 4 | 4 |
| Approximate Viscosity (cP) at 25° C. | NT | ~8860 | ~2780 | ~2900 | ~2600 | ~2820 |
| Density Difference of Aqueous Phase & Oil Phase | NT | 0.07 | 0.064 | 0.12 | 0.09 | 0.07 |
| Stable | Y | Y | Y | N | N | N |

“Y” = Yes,
“N” = No,
“NT” = Not Tested
¹ Examples of miscellaneous ingredients include preservatives, fragrances, pH adjusters, salts, chelating agents, buffers, antioxidants, flavonoids, vitamins, botanical extracts, UV filtering agents, proteins, protein hydrolysates and/or isolates, fillers (e.g., organic and/or inorganic fillers such as talc, calcium carbonate, silica, etc.) and composition colorants.

### Example 2

### Stability Testing

The compositions of Example 1 were evaluated for stability. After preparing the compositions, they were placed in airtight glass jars and maintained at a temperature of 45° C. for 24 hours. After 24 hours, the compositions were visually inspected for phase separation. Compositions C, D, and F phase separated. Compositions A and B, however, did not phase separate.

Without being bound by any particular theory, applicant believes that the density difference between the aqueous phase (water or water-based ingredients) and the oil phase (oils, lipids, emollients) is important to the formation and stability of compositions A and B. The behavior of these two phases, in terms of their relative densities, dictates how effectively they mix and remain mixed over time. A significant density difference between the water and oil phases can lead to phase separation.

### Example 3

### Performance Testing

A commercial benchmark product, inventive composition A, and comparative compositions B, C, and E were evaluated for performance. Composition D was not tested because it suffered severe phase separation. The commercial benchmark product lists the following ingredients:
Water (aqua), dipropylene glycol, polysilicone-29, silicone quaternium-18, glycerin, trideceth-6, Chamomilla recutita (matricaria) flower extract, Calendula officinalis flower extract, potassium sorbate, phenoxyethanol, trideceth-12, sodium benzoate, chlorphenesin, potassium benzoate, disodium EDTA, citric acid, and F #703-2.

Caucasian virgin hair swatches (Type Curl Pattern 2) were washed with a standard shampoo, and excess water was removed after cleansing. One hair swatch was set aside as a control, while the remaining swatches were treated separately with one of the compositions from Example 1. Equal amounts of each composition were applied to damp hair, massaged in, and blow dried. The swatches were then hung in a humidity chamber at 25° C. and 80% relative humidity (RH) for 12 hours. Afterward, the swatches were photographed and assessed for frizz, smoothness, fiber alignment, and shine.

The hair swatch treated with the commercial benchmark product showed no significant difference compared to the control swatch. All compositions (A, B, C, E, and F) performed better than the commercial benchmark and the control. Composition B outperformed compositions C and E, showing less frizz, more smoothness, better fiber alignment, and more shine. Surprisingly, inventive composition A outperformed composition B, and therefore, also outperformed compositions C, E, the commercial benchmark, and the control. The results are summarized as follows:

$$A > B > C \& E > Benchmark > Control$$

Inventive composition A contains fatty acids, which are absent in the commercial benchmark and compositions B-E. It is believed that the inclusion of fatty acids contributes to the stability of the composition and enhances the shine.

Composition B contains guar hydroxypropyltrimonium chloride, which provides a stabilizing effect through its hydrophilic-lipophilic balance, helping to stabilize emulsions and improve viscosity by suspending ingredients evenly. It also reduces interfacial tension and forms a protective film, further improving the formulation's stability.

Unlike the other compositions, inventive composition A does not include guar hydroxypropyltrimonium chloride or other thickening agents typically used to enhance stability. Its unique inclusion of fatty acids sets it apart from the others. Based on the data, it is believed that the fatty acids in composition A contribute to the composition's surprising stabilizing effect, as well as its superior ability to control frizz, provide smoothness, improve fiber alignment, and enhance shine more effectively than all other compositions tested.

The terms “comprising,” “having,” and “including” are used in their open, non-limiting sense.

The terms “a” and “the” are understood to encompass the plural as well as the singular.

All percentages, parts and ratios herein are based upon the total weight of the compositions of the present disclosure, unless otherwise indicated.

The compositions and methods of the present disclosure can comprise, consist of, or consist essentially of the essential elements and limitations of the disclosure described herein, as well as any additional or optional ingredients, components, or limitations described herein or otherwise useful.

The hair treatment composition of the instant disclosure may be a rinse-off composition nor a leave-on composition.

The term “rinse-off” as used herein indicates that the composition is used in a context whereby the composition is ultimately rinsed or washed from the treated surface, (e.g., skin, hair, or hard surfaces) either after or during the application of the product. These rinse-off compositions are to be distinguished from compositions referred to a “leave-on” compositions. For example, a rinse-off composition is applied to the hair, optionally allowed to remain on the hair for a short time (e.g., a few second up to about 5, 10, or 15 minutes) and subsequently rinsed from the hair before the hair styled.

The compositions described throughout this disclosure may be a “leave-on” composition. A “leave-on” (also called leave-in) composition refers to a composition that is applied to skin or hair and is not subjected to immediate rinsing and/or washing, for example for at least 4 hours or for a period of time ranging from 4 hours up to 72 hours, from 4 hours up to 48 hours, or from 8 hours up to 36 hours, or from 8 hours up to 24 hours. In other words, the product is applied to the hair and remains on the hair, as styled, i.e., it is not removed from the hair prior to styling the hair.

The term “non-linear fatty acids” as used in the instant disclosure refers to unsaturated fatty acid and/or branched fatty acids.

The term “particulate filler” refers to particulates that remain as particulates in the hair treatment compositions. They are not dissolved or solubilized by the hair treatment composition. They typically have an average diameter of 20 nm to about 15 μm, about 100 nm to about 12 μm, about 500 nm to about 10 μm, or about 1 μm to about 5 μm, or about 10 nm to about 400 nm.

The hair treatment composition may be transparent, translucent, or opaque.

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure. A human can typically see through a transparent composition, for example, and read the text on the other side of a clear glass or clear plastic bottle containing the composition.

The term “translucent” with respect to a translucent composition indicates that the composition has a transmittance of at least 50% at a wavelength of 600 nm, for example measured using a Lambda 40 UV-visible spectrometer. A human cannot likely see through a translucent composition, for example, and read the text on the other side of a clear glass or clear plastic bottle containing the composition. Rather, the text is usually blurred and difficult or not possible to read, yet movement and structure can normally be identified.

The term “opaque” with respect to an opaque composition indicates that the composition is not transparent or translucent, i.e., has a transmittance of less than 50% at a wavelength of 600 nm, for example measured using a Lambda 40 UV-visible spectrometer.

A “composition colorant” is a compound or ingredient that colors the composition but does not have an appreciable coloring effect on hair. In other words, the composition colorant is included to provide a color to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair. Thus, a composition colorant is different from a hair coloring agent. A composition colorant colors the compositions; a “hair coloring agent,” on the other hand, may change the color of the compositions (and often does) but also changes the color of hair

All ranges and values disclosed herein are inclusive and combinable. For examples, any value or point described herein that falls within a range described herein can serve as a minimum or maximum value to derive a sub-range. Furthermore, all ranges provided are meant to include every specific range within, and combination of sub-ranges between, the given ranges. Thus, a range from 1-5, includes specifically 1, 2, 3, 4 and 5, as well as sub-ranges such as 2-5, 3-5, 2-3, 2-4, 1-4, etc.

The expression “inclusive” for a range means that the limits of the range are included in the defined interval.

Other than in the operating examples, or where otherwise indicated, all numbers expressing quantities of ingredients and/or reaction conditions are to be understood as being modified in all instances by the term “about,” meaning within +/−10% of the indicated number. Thus, and amount of “about 5 wt. %” includes amounts from 4.5 to 5.5 wt. %. An amount of “about 65 wt. %” includes amounts from 58.5 to 71.5 wt. %.

As used herein, the expression “at least one” is interchangeable with the expression “one or more” and thus includes individual components as well as mixtures/combinations.

When referencing amounts “less than” a specified value, the amount includes zero (none). For example, an amount of “less than 5 wt. %” of a specific material means that the specific material may be present, provided it is present in an amount that is below 5 wt. %, or may be absent.

The term “substantially free” or “essentially free” as used herein means that there is less than about 5 wt. % of a specific material added to a composition, based on the total weight of the compositions. Nonetheless, the compositions may include less than about 2 wt. %, less than about 1 wt. %, less than about 0.5 wt. %, less than about 0.1 wt. %, less than 0.01 wt. %, or none of the specified material. For instance, the compositions of the instant disclosure are “essentially anhydrous.” Therefore, the composition includes no water or an amount of water less than about 5 wt. %. Nonetheless, the compositions may include less than about 2 wt. %, less than about 1 wt. %, less than about 0.5 wt. %, less than about 0.1 wt. %, less than 0.01 wt. %, or no water.

The term “active material” as used herein with respect to the percent amount of an ingredient or raw material, refers to 100% activity of the ingredient or raw material. All ingredients are listed as 100% active material unless otherwise indicated.

“Cosmetically acceptable” means that the item in question is compatible with a keratinous substrate such as skin and hair. For example, a “cosmetically acceptable carrier” means a carrier that is compatible with a keratinous substrate such as skin and hair.

Throughout the disclosure, the term “a combination thereof” (or a mixture thereof) may be used following a list of elements as shown in the following example where letters A-F represent the elements: “one or more elements selected from A, B, C, D, E, F, or a combination thereof.” The term, “a combination thereof” does not require that the combination include all of A, B, C, D, E, and F (although all of A, B, C, D, E, and F may be included). Rather, it indicates that a combination of any two or more of A, B, C, D, E, and F can be included. In other words, it is like the phrase “one or more elements selected from A, B, C, D, E, F, and a combination of any two or more of A, B, C, D, E, and F.”

The term “a salt thereof” also relates to “salts thereof.” Thus, where the disclosure refers to “an element selected from A, B, C, D, E, F, a salt thereof, or a combination thereof,” it indicates that that one or more of A, B, C, D, and F may be included, one or more of a salt of A, a salt of B, a salt of C, a salt of D, a salt of E, and a salt of F may be included, or a combination of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counter-ion. This list of counter-ions, however, is nonlimiting.

Some of the compounds discussed throughout the disclosure may be in the form of a salt in the composition or added to the composition in the form of a salt (and may dissociate in the composition). Thus, all compounds and amounts of compounds set forth throughout this disclosure relate to both the salt form of the compound and to the disassociated form of the compound. In other words, even if the expression “a salt thereof” is not specifically or expressly stated with respect to ingredients that can form salts or are available as salts, it is understood that the salt form of the compound is encompassed.

“Volatile”, as used herein, means having a flash point of less than about 100° C.

“Non-volatile”, as used herein, means having a flash point of greater than about 100° C.

The term “polymers,” as defined herein, include homopolymers and copolymers formed from at least two different types of monomers.

The term “INCI” is an abbreviation of International Nomenclature of Cosmetic Ingredients, which is a system of names provided by the International Nomenclature Committee of the Personal Care Products Council to describe personal care ingredients.

All components and elements positively set forth in this disclosure can be negatively excluded from the claims. In other words, the compositions of the instant disclosure can be free or essentially free of any components and elements positively recited throughout the instant disclosure.

Some of the various categories of components identified may overlap. In such cases where overlap may exist and the composition includes both components (or the composition includes more than two components that overlap), an overlapping component or ingredient does not represent more than one element of the claims. For example, if a polyacrylate falls within the description of a thickening polymer and within the description of film forming polymer, a single polyacrylate is understood as being only the thickening polymer or only the film forming polymer. A single polyacrylate cannot simultaneously be construed as both a thickening polymer and a film forming polymer in the same composition, for example, when the claims set forth both a thickening polymer and a film forming polymer (even though the polyacrylate may function as both a thickening polymer and a film forming polymer). In other words, a single compound or ingredient cannot simultaneously serve as two different components or elements of a claim.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair treatment composition comprising:
(a) about 0.1 to about 8 wt. % of one or more cationic amidoamine surfactants;
(b) about 0.1 to about 5 wt. % of one or more sebacic acid copolymers;
(c) about 0.1 to about 10 wt. % of one or more vegetable oils;
(d) about 0.1 to about 10 wt. % of one or more fatty acids;
(e) optionally, one or more fatty alcohols;
(f) about 5 to about 25 wt. % of one or more polyols with 2 to 8 carbon atoms;
(g) optionally, about 0.01 to about 5 wt. % of one or more cationic polymers; and
(h) water;
wherein the hair treatment composition is an oil-in-water emulsion, and all percentages by weight are based on a total weight of the hair treatment composition.

2. The composition of claim 1 wherein the one or more cationic amidoamine surfactants are selected from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamidopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, and stearamidoethyldiethylamine.

3. The composition of claim 1, wherein the one or more sebacic acid copolymers are selected from capryloyl glycerin/sebacic acid copolymer, hydrogenated castor oil/sebacic acid copolymer, polyhydroxybutyrate-sebacate copolymer, acrylates/sebacic acid copolymer, polycaprolactone/sebacic acid copolymer, polylactic acid/sebacic acid copolymer, poly(sebacic acid-co-ethylene glycol), poly(sebacic acid-co-1,4-butanediol), and poly(sebacic acid-co-lactic acid).

4. The composition of claim 1, wherein the one or more vegetable oils are selected from castor oil, arachis oil, amaranth seed oil, apricot oil, argan oil, avocado oil, babassu oil, cottonseed oil, borage seed oil, camelina oil, camellia oil, safflower oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, manila oil, evening primrose oil, olive oil, palm oil, palm kernel oil, brazil nut oil, pecan nut oil, peach kernel oil, rapeseed oil, rosehip oil, sea buckthorn fruit oil, sea buckthorn kernel oil, sesame oil, soybean oil, sunflower oil, grapeseed oil, walnut oil, wild rose oil, wheat germ oil, coconut oil, avocado oil, arachis oil, apricot oil, argan oil, and babassu oil.

5. The hair treatment composition of claim 1, wherein the one or more fatty acids are selected from myristic acid, lauric acid, palmitic acid, stearic acid, behenic acid, arichidonic acid, oleic acid, isostearic acid, and sebacic acid.

6. The hair treatment composition of claim 1, wherein the one or more polyols with 2 to 8 carbon atoms are selected from ethylene glycol, propylene glycol, butylene glycol, hexylene glycol, pentylene glycol, 1,3-propanediol, diethylene glycol, dipropylene glycol, caprylyl glycol, and glycerin.

7. The hair treatment composition of claim 1 comprising the one or more fatty alcohols.

8. The hair treatment composition of claim 7, wherein the one or more fatty alcohols are selected from octyldodecanol, decyl alcohol, undecyl alcohol, dodecyl alcohol, myristyl alcohol, cetearyl alcohol, cetyl alcohol, stearyl alcohol, isostearyl alcohol, isocetyl alcohol, behenyl alcohol, linalool, oleyl alcohol, isotridecyl alcohol, and myricyl alcohol.

9. The hair treatment composition of claim 1 comprising about 65 to about 90 wt. % of the water.

10. The hair treatment composition of claim 1, further comprising one or more cationic polymers.

11. The hair treatment composition of claim 9, wherein at least one of the one or more cationic polymers is a cationic polysaccharide selected from cationic celluloses, cationic starches, cationic dextrans, and cationic guars.

12. The hair treatment composition of claim 11, wherein the cationic polysaccharide is a cationic guar selected from guar hydroxypropyltrimonium chloride, hydroxypropyl guar hydroxypropyltrimonium chloride, guar dimethylhydroxypropyltrimonium chloride, guar hydroxypropyltrimonium methylsulfate, or mixtures thereof.

13. The hair treatment composition of claim 1, wherein the hair treatment composition has a pH of about 2.5 to about 5.

14. The hair treatment composition of claim 1, wherein the hair treatment composition has a viscosity of about 200 to about 50,000 cP at 25° C.

15. The hair treatment composition of claim 1, wherein the oil-in-water emulsion has a density difference between its aqueous phase and its oil phase of less than 0.07.

16. The hair treatment composition of claim 1, wherein (a), (b), (c), and (d) are in a combined amounts of about 2 to about 10 wt. %.

17. The hair treatment composition of claim 8, wherein (a), (b), (c), (d), and (e) are in a combined amount of about 4 to about 12 wt. %.

18. The hair treatment composition of claim 1, wherein the hair treatment composition is free from silicones.

19. A hair treatment composition comprising:
(a) about 0.1 to about 8 wt. % of one or more cationic amidoamine surfactants;
(b) about 0.1 to about 5 wt. % of hydrogenated castor oil/sebacic acid copolymer;
(c) about 0.1 to about 10 wt. % of castor oil;
(d) about 0.1 to about 10 wt. % of one or more fatty acids;
wherein (a), (b), (c), and (d) are in a combined amounts of about 2 to about 10 wt. %;
(e) one or more fatty alcohols;
wherein (a), (b), (c), (d), and (e) are in a combined amounts of about 4 to about 12 wt. %;
(f) about 5 to about 25 wt. % of one or more polyols with 2 to 10 carbon atoms;
(g) water;
(h) optionally, one or more cationic polysaccharides; and
(i) about 0.1 to about 10 wt. % of one or more miscellaneous ingredients;
wherein the hair treatment composition is an oil-in-water emulsion, and all percentages by weight are based on a total weight of the hair treatment composition.

20. A method for treating hair comprising applying the hair treatment composition of claim 1 to the hair.
